(19) United States
(12) Patent Application Publication
(10) Pub. No.: US 2017/0216214 A1
(43) Pub. Date: Aug. 3, 2017

(54) FORMULATIONS

(71) Applicant: Hoffmann-La Roche Inc., Little Falls, NJ (US)

(72) Inventors: Eric Garcia, Uffholtz (FR); Nicole Kaiser, Loerrach (DE); Joerg Kriese, Basel (CH); Susanne Amrhein, Gelterkinden (CH); Thomas Meyer, Oberwil/BL (CH); Susanne Page, Loerrach (DE)

(73) Assignee: Hoffmann-La Roche Inc., Little Falls, NJ (US)

(21) Appl. No.: 15/486,031

(22) Filed: Apr. 12, 2017

Related U.S. Application Data
(63) Continuation of application No. 14/526,030, filed on Oct. 28, 2014, now abandoned.
Application No. 14/526,030 is a continuation of application No. PCT/EP2013/058685, filed on Apr. 26, 2013.

(30) Foreign Application Priority Data
Apr. 30, 2012 (EP) 12166211.8

(51) Int. Cl. A61K 9/20; A61K 9/28; A61K 31/40

(52) U.S. Cl. CPC A61K 9/2054; A61K 31/40; A61K 9/2013; A61K 9/284; A61K 9/2866; A61K 9/2813

(57) ABSTRACT

The present invention relates to a hygroscopic matrix based composition, a process for the preparation thereof and its use in the treatment of diseases.

### CROSS-REFERENCE TO RELATED APPLICATIONS

This application is a Continuation of U.S. application Ser. No. 14/526,030 filed on Oct. 28, 2014, which is a Continuation of PCT/EP2013/058685 filed on Apr. 26, 2013, which is entitled to the priority of EP Application No. 12166211.8 filed on Apr. 30, 2012.

### FIELD OF THE INVENTION

The present invention relates to a fixed dose combination formulation, a process for the preparation thereof and its use in the treatment of diseases.

### BACKGROUND OF THE INVENTION

Active pharmaceutical ingredients showing an ester, amide or thioester functionality, such as S-[2-([[1-(2-ethylbutyl)cyclohexyl]carbonyl]amino) phenyl] 2-methylpropanethioate, are often sensitive to moisture and frequently show chemical incompatibility with a wide range of commonly used pharmaceutical excipients, thus typical composition approaches such as lipid based drug delivery systems can not be considered. Incorporating the drug substance into a hygroscopic polymer matrix can be critical due to chemical as well as physical stability. The sorption of moisture by excipients in solid dosage forms can lead to considerable stability problems when the contained active pharmaceutical is instable in water due to the presence of a hydrolysis sensitive functional group. Though theoretically hygroscopic polymers are capable to bind moisture in the composition, thus protecting the active pharmaceutical ingredient from hydrolysis, a fairly high amount of polymer is needed to achieve this, what usually leads to capping or cracking of the immediate-release tablet composition. Thus, it is usually imperative to prevent moisture sorption during storage by both, a suitable composition and primary packaging.

### SUMMARY OF THE INVENTION

The manufacturing of the composition according to the present invention shows surprisingly better flowability than past compositions comprising a hydrophobic, water instable compound with a waxy consistency. For instance, the composition according to the present invention does not demonstrate extreme funnel flow.

In a first aspect the present invention provides a composition comprising S-[2-([[1-(2-ethylbutyl)cyclohexyl]carbonyl]amino) phenyl] 2-methylpropanethioate (a hydrophobic, water instable compound with a waxy consistency), a super-disintegrant and atorvastatin.

In a second aspect the present invention provides a composition comprising S-[2-([[1-(2-ethylbutyl)cyclohexyl]carbonyl]amino) phenyl] 2-methylpropanethioate (a hydrophobic, water instable compound with a waxy consistency), a super-disintegrant, atorvastatin and at least two diluents with a bulk density lower than 800 g/L.

The invention also provides a method for treating or preventing a cardiovascular disorder in a mammal by administering to a mammal in need of such treatment a therapeutically effective amount of the composition provided by the invention.

The invention further provides a composition for treating or preventing a cardiovascular disorder. A composition according to the present invention for the use in the treatment or prevention of cardiovascular disorder is also part of the invention.

The hygroscopic matrix based composition is useful to chemically stabilize a hydrophobic and hydrolysis sensitive compound with a waxy consistency, such as S-[2-([[1-(2-ethylbutyl)cyclohexyl]carbonyl]amino) phenyl] 2-methylpropanethioate and to stabilize the physical properties of a tablet comprising said composition.

### BRIEF DESCRIPTION OF THE FIGURES:

FIG. 1 is a 3D reconstruction of all X-ray slices of a tablet produced according to example 1.

FIG. 2 is a 3D reconstruction of all X-ray slices of a tablet according to placebo example A.

FIG. 3 illustrates a X-ray powder diffraction pattern of S-[2-([[1-(2-ethylbutyl)cyclohexyl]carbonyl]amino) phenyl] 2-methylpropanethioate crystalline form, also known as Form A.

FIG. 4 is a schematic drawing of the composition according to the present invention which comprises the core comprising dalcetrapib (A), the separation layer (B), the active coating comprising atorvastatin (C) and a seal coating or film coating (D).

FIG. 5 is a schematic drawing of the composition according to the present invention which comprises the one layer comprising dalcetrapib (A), another layer comprising atorvastatin (B) and a seal coating or film coating (C).

### DETAILED DESCRIPTION OF THE INVENTION

Unless otherwise stated, the following terms used in the specification and claims have the meanings given below:

The term “bulk density” refers to a density measurement of a loose, uncompacted substance, wherein the volume of the substance includes the air trapped between particles. The bulk density is measured in a graduated cylinder according to the European Pharmacopeia.

The term “diluent” refers to an excipient which fills out the size of a tablet or capsule, making it practical to produce and convenient for the consumer to use. Suitable diluents include e.g. pharmaceutically acceptable fillers, such as microcrystalline cellulose (e.g. Avicel®), crospovidone micronized, cellulose powder, lactose spray-dried, lactose anhydrous, lactose monohydrate, dibasic calcium phosphate, sugars, sugar alcohols, corn starch, starch, pregelatinized starch, colloidal silicon dioxide, polysaccharides, and mixtures thereof.

The term “hydrophobic” means insoluble in water, not readily absorbing moisture, or being adversely affected by water; either incompatible with water or having little affinity for it. In other words the hydrophobic drug or compound would not spontaneously disperse in water. Specifically hydrophobic means logP>3. The logP is measured or in the absence of experimental data calculated as clogP according to the model developed by Moriguchi (S. Moriguchi, S. Hirono, I. Nakagome, H. Hirano, (1994). “Comparison of reliability of log P values for drugs calculated by several methods” Chem Pharm Bull 1994, 42: 976-978).

The term “hygroscopic polymeric excipient(s)” means polymeric excipient(s) which take(s) up moisture for example by absorption or adsorption even at relative humidity as low as 50%, at room temperature (e.g. about 25° C.). The moisture uptake is measured e.g by dynamic vapor sorption at room temperature. As an example the hygroscopicity can be measured in accordance with the method disclosed in the European Pharmacopoeia—6th Edition (2008), Chapter 5.11. The dynamic vapor sorption technique measures the change in mass which is produced by varying the vapor concentration surrounding the product. Suitable “hygroscopic polymeric excipients” are hydroxypropyl methylcellulose, hydroxypropyl cellulose, low-substituted hydroxypropyl cellulose, hydroxyethylmethyl cellulose, carboxypolymethylene, methylcellulose, ethylcellulose, hydroxyethyl cellulose, celluloseacetate, polyvinylpyrrolidone crosslinked polyvinylpyrrolidone, micronized crosslinked polyvinylpyrrolidone, carboxymethylcellulose sodium, carboxymethylcellulose calcium, crosslinked carboxymethylcellulose, microcrystalline cellulose, silicified microcrystalline cellulose, cellulose powder, carboxymethyl starch, starch, pregelatinized starch or mixture thereof. In particular “hygroscopic polymeric excipients” refer to hydroxypropyl methylcellulose, carboxymethylcellulose sodium, microcrystalline cellulose and micronized crosslinked polyvinylpyrrolidone. Examples of “water insoluble hygroscopic polymers” at room temperature (e.g. about 25° C.) include low-substituted hydroxypropyl cellulose, carboxypolymethylene, ethylcellulose, celluloseacetate, crosslinked polyvinylpyrrolidone, micronized crosslinked polyvinylpyrrolidone, carboxymethylcellulose calcium, microcrystalline cellulose, silicified microcrystalline cellulose, cellulose powder, and starch.

The term “Super-disintegrant” refers to disintegrants that very rapidly expand upon contact with water, Generally speaking, superdisintegrants are disintegration agents which can be used in a fractional amount of normal disintegrants to obtain the same effect. Examples of superdisintegrants include cross-linked carboxymethyl cellulose sodium (a.k.a. croscarmellose sodium), sodium starch glycolate, and cross-linked polyvinyl pyrollidone (a.k.a. crospovidone). Croscarmellose sodium is commercially available from FMC Corp. under the trade name Ac-Di-Sol® and from Avebe Corp. under the trade name Primellose®®. Sodium starch glycolate is commercially available from Penwest Pharmaceuticals Co. under the tradename Explotab® and from Avebe Corp. under the tradename Primojel®. Crospovidone is commercially available from BASF Corp. under the tradename Kollidon® CL and from International Specialty Chemicals Corp. under the tradename Polyplasdone®. Croscarmellose is also commercially available from Mingtai Chemical Co. Ltd under the tradename DISOLCEL® and from J. Rettenmaier & Söhne GmbH+Co (JRS) under the tradename Vivasol®. The most preferred superdisintegrants are croscarmellose sodium and crospovidone.

The term “water instable” means the presence of a hydrolysis sensitive functional group like an ester, amide or thioester.

The term “waxy consistency” means that the glass transition temperature (Tg) is lower than 25° C.

The term “pharmaceutical acceptable metal salt” refers to sodium, potassium, lithium, calcium, magnesium, aluminum, iron, or zinc salts.

The term “PVA” and “PVOH” are interchangeable and refer to a polyvinyl alcohol which in particular are polyvinyl resins having hydroxyl groups and is obtained through saponification of polyvinyl acetate (polymerized vinyl acetate). More particularly the polyvinyl alcohol are obtained from Nippon-Gohsei (Gohsenol).

S-[2-([[1-(2-ethylbutyl)cyclohexyl]carbonyl]amino) phenyl] 2-methylpropanethioate is also known as thioisobutyric acid S-(2-{[1-(2-ethyl-butyl)-cyclohexanecarbonyl]-amino}-phenyl) ester, dalcetrapib or a compound of formula I

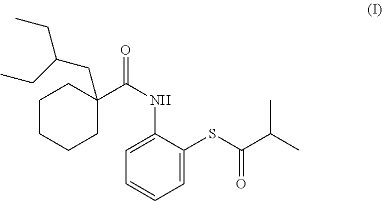 [SMILES: CCC(CC)CC1(C(=O)Nc2ccccc2SC(=O)C(C)C)CCCCC1]

Atorvastatin refers to atorvastatin pharmaceutically acceptable salts and/or hydrates also known as [R-(R*,R*)]-2-(4-fluorophenyl)-β,δ-dihydroxy-5-(1-methylethyl)-3-phenyl-4-[(phenylamino)carbonyl]-1H-pyrrole-1-heptanoic acid, (2R-trans)5-(4-fluorophenyl)-2-(1-methyl ethyl)-N,4-diphenyl-1-[2-(tetrahydro-4-hydroxy-6-oxo-2Hpyran-2-yl)ethyl-1H-pyrrole-3-carboxamide, atorvastatin acid or a compound of formula (II′)

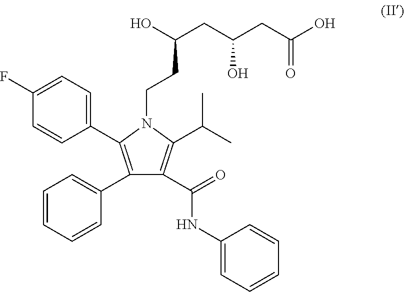 [SMILES: CC(C)c1c(C(=O)Nc2ccccc2)c(-c2ccccc2)c(-c2ccc(F)cc2)n1CC[C@@H](O)C[C@@H](O)CC(=O)O]

pharmaceutical acceptable salts and/or hydrates. Atorvastatin refers in particular to [R-(R*,R*)]-2-(4-fluorophenyl)-β,δ-dihydroxy-5-(1-methylethyl)-3-phenyl-4-[(phenyl amino)carbonyl]-1H-pyrrole-1-heptanoic acid pharmaceutically acceptable salts and/or hydrate. The pharmaceutically salt is selected from monosodium salt, monopotassium salt, hemicalclium salt, N-methylglucamine salt, hemimagnesium salt or hemizinc salt, in particular hemicalclium salt or hemimagnesium salt, more particularly hemicalclium salt. More particularly atorvastatin refers to hemicalcium salt of [R-(R*,R*)]-2-(4-fluorophenyl)-β,δ-dihydroxy-5-(1-methylethyl)-3-phenyl-4-[(phenylamino)carbonyl]-1H-pyrrole-1-heptanoic acid also known [R-(R*,R*)]-2-(4-fluorophenyl)-β,δ-dihydroxy-5-(1-methylethyl)-3-phenyl-4-[(phenylamino)carbonyl]-1H-pyrrole-1-heptanoic acid hemicalcium salt or [R-(R*,R*)]-2-(4-fluorophenyl)-β,δ-dihydroxy-5-(1-methylethyl)-3-phenyl-4-[(phenylamino)carbonyl]-1H-pyrrole-1-heptanoic acid, calcium salt (2:1). Its chemical structure may be represented by formula (II):

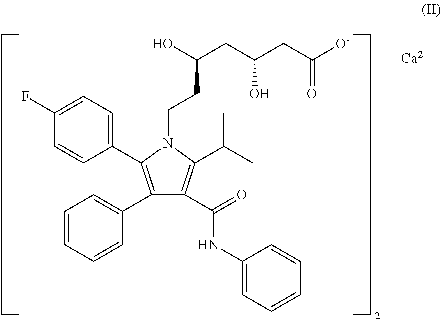

Even more particularly atorvastatin refers to [R-(R*,R*)]-2-(4-fluorophenyl)-β,δ-dihydroxy-5-(1-methylethyl)-3-phenyl-4-[(phenylamino)carbonyl]-1H-pyrrole-1-heptanoic acid, calcium salt (2:1) trihydrate, most particularly in crystalline form I as disclosed in WO9703959. Form I is characterized by an X-ray powder diffraction pattern having peaks at about 9.2, 9.5, 10.3, 10.6, 11.9, 12.2, 17.2, 19.5, 21.6, 22.0, 22.7, 23.3, 23.7, 24.4, 28.9 and 29.2±0.2°, particularly by an XRPD peaks observed at an angle of diffraction 2Theta of 11.9, 17.1 and 21.6 (±0.2°).

Atorvastatin calcium is currently been sold as Lipitor®. Lipitor Atorvastatin has been described in EP1061073 B1, EP0409281 B1, EP0848705B1, EP 1148049 B1, EP0247633B1 and WO9416693.

Atorvastatin is a synthetic reversible inhibitor of the microsomal enzyme HMG-CoA reductase. Atorvastatin is usually administred orally as the calcium salt of the active hydroxyl acid in a dosage range of 10-80 mg/day. Atorvastatin acid is converted to its lactone in vivo in humans, and these two forms appear to have approximately the same AUC (Area Under the Curve).

Lipitor° tablets for oral administration comprise 10 mg, 20 mg, 40 mg or 80 mg atorvastatin and the following excipients: calcium carbonate, croscarmellose sodium, hydroxypropyl cellulose, hydroxypropyl methylcellulose, lactose monohydrate, magnesium stearate, microcrystalline cellulose, polyethylene glycol, polysorbate 80, simethicone emulsion, talc, and titanium dioxide. In addition Lipitor® may comprise candelilla wax.

Atorvastatin is unstable, as it is susceptible to heat, moisture, low pH environment and light. In acidic environment, atorvastatin will degrade to lactone. Furthermore, atorvastatin will decompose rapidly when exposed to UV or fluorescent lights. Atorvastatin may be destabilized by contact with molecular moieties of other components such excipients used in the core layer, dal layer or atv layer, or/and dalcetrapib. Therefore a stabilizing means may be required for effective pharmaceutical dosages.

In another embodiment of the present invention, at least one pharmaceutically acceptable stabilizing additive is present. In particular, the pharmaceutically acceptable stabilizing additive would be in close vicinity to atorvastatin. More particularly, the pharmaceutically acceptable stabilizing additive is present in the active coating comprising atorvastatin or in the atv layer. In particular the pharmaceutically acceptable stabilizing additive is selected from alkaline earth metal salts such as, calcium carbonate, calcium hydroxide, magnesium carbonate, magnesium carbonate, magnesium hydroxide, magnesium silicate, magnesium aluminate or aluminum magnesium hydroxide, or a mixture thereof. More particularly the pharmaceutically acceptable stabilizing additive is calcium carbonate.

In another embodiment of the invention, polyethylene glycol is not present in the active coating.

According to the present invention, the composition needs to maintain a good dissolution rate of atorvastatin and dalcetrapib, in particular similar to the mono-therapy compositions of atorvastatin and dalcetrapib. In particular, the composition according to the present invention produces similar exposures of dalcetrapib and atorvastatin to the mono-therapy reference tablets.

More particularly, according to a more particular embodiment of the present invention, the composition shows similar impurities profile to the mono-therapy reference tablets, atorvastatin and dalcetrapib.

Calcium carbonate has some incompatibilities with dalcetrapib and/or with some of the impurities of dalcetrapib. It increases the formation of iso-butyric acid which in turn increases the atorvastatin lactone formation.

When calcium carbonate is not present in the active coating, the dissolution rate of atorvastatin increases, which in turn increases the dalcetrapib dissolution.

Unless otherwise stated all percentages are given in weight percent of the total weight of the composition.

S-[2-([[1-(2-ethylbutyl)cyclohexyl]carbonyl]amino) phenyl] 2-methylpropanethioate has been shown to be an inhibitor of CETP activity in humans (de Grooth et al., Circulation, 105, 2159-2165 (2002)) and rabbits (Shinkai et al., J. Med. Chem., 43, 3566-3572 (2000); Kobayashi et al., Atherosclerosis, 162, 131-135 (2002); and Okamoto et al., Nature, 406 (13), 203-207 (2000)). S-[2-([[1-(2-ethylbutyl) cyclohexyl]carbonyl]amino) phenyl] 2-methylpropanethioate has been shown to increase plasma HDL cholesterol in humans (de Grooth et al., supra) and in rabbits (Shinkai et al., supra; Kobayashi et al., supra; Okamoto et al., supra). Moreover, S-[2-([[1-(2-ethylbutyl) cyclohexyl]carbonyl]amino) phenyl] 2-methylpropanethioate has been shown to decrease LDL cholesterol in humans (de Grooth et al. , supra) and rabbits (Okamoto et al., supra). Additionally, S-[2-([[1-(2-ethylbutyl)cyclohexyl]carbonyl]amino)phenyl] 2-methylpropanethioate inhibits the progression of atherosclerosis in rabbits (Okamoto et al., supra). S-[2-([[1-(2-ethylbutyl)cyclohexyl]carbonyl]amino) phenyl] 2-methylpropanethioate, as well as methods of making and using the compound, are described in EP patent EP1020439, Shinkai et al., J. Med. Chem. 43:3566-3572 (2000) or WO 2007/051714, WO 2008/074677 or WO2011/000793.

In a particular embodiment S-[2-([[1-(2-ethylbutyl)cyclohexyl]carbonyl]amino) phenyl] 2-methylpropanethioate is a solid in crystalline or amorphous form, more particularly in crystalline form. In a particular embodiment S-[2-([[1-(2-ethylbutyl)-cyclohexyl]-carbonyl]amino)phenyl]2-methylpropanethioate is in crystalline form A.

Form A is characterized by an X-ray powder diffraction pattern having peaks at about 7.9°, 8.5°, 11.7°, 12.7°, 17.1°, 18.0°, 18.5°, 20.2°, 22.1°, 24.7°±0.2°, particularly by an XRPD peaks observed at an angle of diffraction 2Theta of 7.9°, 11.7°, 17.1°, 18.5° (±0.2°).

The composition can be used to treat or prevent a cardiovascular disorder, including, but not limited to, atherosclerosis, peripheral vascular disease, dyslipidemia (e. g., hyperlipidimia), hyperbetalipoproteinemia, hypoalphalipoproteinemia, hypercholesterolemia, hypertriglyceridemia, familial-hypercholesterolemia, angina, ischemia, cardiac ischemia, stroke, myocardial infarction, reperfusion injury, angioplastic restenosis, hypertension, cardiovascular disease, coronary heart disease, coronary artery disease, acute coronary syndrome, hyperlipidoproteinemia, vascular complications of diabetes, obesity or endotoxemia in a mammal, especially a human (i. e., a male or female human). The composition can be used to reduce cardiovascular morbidity and mortality.

Accordingly, the invention provides a method for the treatment or prophylaxis of a cardiovascular disorder in a mammal, which method comprises administering to a mammal (particularly a mammal in need thereof) a therapeutically effective amount of the composition. The mammal particularly is a human (i. e., a male or female human). The human can be of any race (e. g., Caucasian or Oriental). The cardiovascular disorder particularly is selected from the group consisting of atherosclerosis, peripheral vascular disease, dyslipidemia, hyperbetalipoproteinemia, hypoalphalipoproteinemia, hypercholesterolemia, hypertriglyceridemia, familial-hypercholesterolemia, angina, ischemia, cardiac ischemia, stroke, myocardial infarction, reperfusion injury, angioplastic restenosis, hypertension, and vascular complications of diabetes, obesity or endotoxemia in a mammal. More particularly, the cardiovascular disorder is selected from the group consisting of cardiovascular disease, coronary heart disease, coronary artery disease, acute coronary syndrome, hypoalphalipoproteinemia, hyperbetalipoproteinemia, hypercholesterolemia, hyperlipidemia, atherosclerosis, hypertension, hypertriglyceridemia, hyperlipidoproteinemia, peripheral vascular disease, angina, ischemia, and myocardial infarction.

In particular embodiment of the invention, the composition comprises: a) a core comprising S-[2-([[1-(2-ethylbutyl)cyclohexyl]carbonyl]amino) phenyl] 2-methylpropanethioate and b) an active coating comprising atorvastatin or the composition comprises: a) one layer comprising S-[2-([[1-(2-ethylbutyl)cyclohexyl]carbonyl]amino) phenyl] 2-methylpropanethioate (herein referred as dal layer) and b) another layer comprising atorvastatin (herein referred as atv layer). In particular, the active coating comprising atorvastatin would not be in contact with S-[2-([[1-(2-ethylbutyl)cyclohexyl]carbonyl]amino) phenyl] 2-methylpropanethioate.

In a particular embodiment of the invention, the composition is a fixed dose tablet, particularly in the form of a bilayer tablet or in an active coating tablet.

In certain embodiments of the present invention, the composition comprises: 10% to 69% by weight of the total weight of the core or dal layer, particularly 40% to 60% by weight of the total weight of the core or dal layer, more particularly 48% to 55% by weight of the total weight of the core or dal layer of S-[2-([[1-(2-ethylbutyl)cyclohexyl]carbonyl]amino) phenyl] 2-methylpropanethioate.

In certain embodiments of the present invention, the composition comprises: 1% to 10% by weight of the total weight of the core or dal layer, particularly 5% to 10% by weight of the total weight of the core or dal layer, more particularly 4% to 8% by weight of the total weight of the core or dal layer of a super-disintegrant.

In certain embodiments of the present invention, the composition comprises 30% to 70% by weight of the total weight of the core or dal layer, particularly 30% to 60% by weight of the total weight of the core or dal layer, more particularly 40% to 50% by weight of the total weight of the core or dal layer of at least two diluents with a bulk density lower than 800 g/L.

In a particular embodiment, the present invention provides a composition comprising:
a) 10% to 69% by weight of the total weight of the core or dal layer, particularly 40% to 60% by weight of the total weight of the core or dal layer, more particularly 48% to 55% by weight of the total weight of the core or dal layer of S-[2-([[1-(2-ethylbutyl)cyclohexyl]carbonyl]amino) phenyl] 2-methylpropanethioate
1% to 10% by weight of the total weight of the core or dal layer, particularly 5% to 10% by weight of the total weight of the core or dal layer, more particularly 4% to 8% of the total weight of the core or dal layer by weight of a super-disintegrant, and
30% to 70% weight of the total weight of the core or dal layer, particularly 30% to 60% by weight of the total weight of the core or dal layer, more particularly 40% to 50% by weight of the total weight of the core or dal layer of at least two diluents with a bulk density lower than 800 g/L; and
b) atorvastatin.

In certain embodiments of the present invention as defined herein, the super-disintegrant is a hygroscopic polymeric excipient. In particular the hygroscopic polymeric excipient as superdisintegrant is croscarmellose sodium.

In a particular embodiment, the present invention provides a composition comprising:
a) S-[2-([[1-(2-ethylbutyl)-cyclohexyl]-carbonyl]amino)phenyl]2-methylpropanethioate;
   croscarmellose sodium; and
b) atorvastatin.

In another embodiment, the present invention provides a composition comprising:
a) a core or a dal layer comprising:
   S-[2-([[1-(2-ethylbutyl)-cyclohexyl]-carbonyl]amino)phenyl]2-methylpropanethioate; and
   croscarmellose sodium; and
b) an active coating or atv layer comprising atorvastatin.

In certain embodiments of the present invention as defined herein, the composition further comprises at least one additional hygroscopic polymeric excipient, in particular in the core or dal layer.

In certain embodiments of the present invention as defined herein, the composition further comprises at least two hygroscopic polymeric excipients, in particular in the core or dal layer.

In certain embodiments of the present invention as defined herein, the composition further comprises at least three hygroscopic polymeric excipients of which two are diluents with a bulk density lower than 800 g/L, in particular in the core or dal layer.

In certain embodiments of the present invention as defined herein, the composition comprises 10% to 69% by weight of the total weight of the core or dal layer of S-[2-([[1-(2-ethylbutyl)-cyclohexyl]-carbonyl]amino)phenyl]2-methylpropanethioate.

In certain embodiments of the present invention, the composition comprises: 10% to 69% by weight of the total weight of the core or dal layer, particularly 40% to 60% by weight of the total weight of the core or dal layer, more particularly 48% to 55% by weight of the total weight of the core or dal layer of S-[2-([[1-(2-ethylbutyl)-cyclohexyl]-carbonyl]amino)phenyl]2-methylpropanethioate.

In certain embodiments of the present invention as defined herein, the composition comprises 1% to 10% by weight of the total weight of the core or dal layer, particularly 5% to 10% by weight of the total weight of the core or dal layer, more particularly 5% to 8% by weight of the total weight of the core or dal layer of croscarmellose sodium. More particularly, in a certain embodiment, the composition comprises 5% to 7% by weight of the total weight of the core or dal layer of croscarmellose sodium.

In certain embodiments of the present invention as defined herein, the composition comprises at least 30% by weight of the total weight of the core or dal layer of the hygroscopic polymeric excipients, in particular 44% to 50% by weight of the total weight of the core or dal layer, more particularly 46% to 48% by weight of the total weight of the core or dal layer, wherein the hygroscopic polymeric excipients are hydroxypropylmethyl cellulose, croscarmellose sodium, microcrystalline cellulose and micronized crosslinked polyvinylpyrrolidone.

In certain embodiments of the present invention as defined herein, the composition comprises at least 30% by weight of the total weight of the core or dal layer of the hygroscopic polymeric excipients, particularly 34% to 44% by weight of the total weight of the core or dal layer, more particularly 40% to 44% by weight of the total weight of the core or dal layer.

In certain embodiments of the present invention as defined herein, the composition comprises at least 30% by weight of the total weight of the core or dal layer of the additional hygroscopic polymeric excipients, particularly 34% to 44% by weight of the total weight of the core or dal layer, more particularly 40% to 44% by weight of the total weight of the core or dal layer.

In a particular embodiment, the present invention provides a composition comprising:
a) 10% to 69% by weight of the total weight of the core or dal layer, particularly 40% to 60% by weight of the total weight of the core or dal layer, more particularly 48% to 55% by weight of the total weight of the core or dal layer of S-[2-([[1-(2-ethylbutyl)-cyclohexyl]-carbonyl]amino)phenyl]2-methylpropanethioate;
1% to 10% by weight of the total weight of the core or dal layer, particularly 5% to 10% by weight of the total weight of the core or dal layer, more particularly 4% to 8% by weight of the total weight of the core or dal layer of croscarmellose sodium, and
30% to 90% by weight of the total weight of the core or dal layer, particularly 34% to 44% by weight of the total weight of the core or dal layer, more particularly 40% to 44% by weight of the total weight of the core or dal layer of the hygroscopic polymeric excipients; and
b) atorvastatin.

wherein the hygroscopic polymeric excipients are selected from hydroxypropylmethyl cellulose, microcrystalline cellulose and micronized crosslinked polyvinylpyrrolidone.

In certain embodiments of the present invention as defined herein, the composition comprises:
a) 48% to 55% by weight of the total weight of the core or dal layer of S-[2-([[1-(2-ethylbutyl)-cyclohexyl]-carbonyl]amino)phenyl]2-methylpropanethioate;
4% to 8% by weight of the total weight of the core or dal layer, of croscarmellose sodium
32% to 41% by weight of the total weight of the core or dal layer of water insoluble hygroscopic polymer; and
4% to 5% by weight of the total weight of the core or dal layer of water soluble hygroscopic polymer; and
b) atorvastatin.

In certain embodiments of the present invention as defined herein, wherein the hygroscopic polymeric excipients are selected from hydroxypropylmethyl cellulose, hydroxypropyl cellulose, low-substituted hydroxypropyl cellulose, hydroxyethylmethyl cellulose, carboxypolymethylene, methylcellulose, ethylcellulose, hydroxyethyl cellulose, celluloseacetate, polyvinylpyrrolidone, crosslinked polyvinylpyrrolidone, micronized crosslinked polyvinylpyrrolidone, carboxymethylcellulose calcium, crosslinked carboxymethylcellulose, microcrystalline cellulose, silicified microcrystalline cellulose, cellulose powder, carboxymethyl starch, starch and pregelatinized starch.

In certain embodiments of the present invention as defined herein, wherein the hygroscopic polymeric excipients are hydroxypropylmethyl cellulose, microcrystalline cellulose and micronized crosslinked polyvinylpyrrolidone.

In certain embodiments of the present invention as defined herein, the two diluents are hygroscopic polymeric excipients. In particular the hygroscopic polymeric excipients as diluents are ethylcellulose, micronized crosslinked polyvinylpyrrolidone, microcrystalline cellulose, silicified microcrystalline cellulose, cellulose powder, starch, pregelatinized starch.

In certain embodiments of the present invention as defined herein, at least two hygroscopic polymeric excipients are present.

In certain embodiments of the present invention as defined herein, the super-disintegrant and at least one of the diluents, or at least two diluents are hygroscopic polymeric excipients. More particularly, at least the super-disintegrant and one of the diluents are hygroscopic polymeric excipients.

In certain embodiments of the present invention as defined herein, the super-disintegrant and the two diluents are hygroscopic polymeric excipients.

In certain embodiments of the present invention as defined herein, there is at least 30% by weight of the total weight of the core or dal layer of hygroscopic polymeric excipients, particularly 44% to 50% by weight of the total weight of the core or dal layer.

In certain embodiments of the present invention, the super-disintegrant is croscarmellose sodium. In particular, the present invention comprises up to 6% by weight of the total weight of the core or dal layer of croscarmellose sodium.

The invention provides a physically stable composition comprising a) at least S-[2-([[1-(2-ethylbutyl)-cyclohexyl]-carbonyl]amino)phenyl]2-methylpropanethioate, a hydrophobic and water instable cholesteryl ester transfer protein (CETP) inhibitor embedded in a chemically protective hygroscopic polymer matrix tablet consisting of at least one hygroscopic polymer e.g. hydroxypropylmethyl cellulose (HPMC), hydroxypropyl cellulose (HPC), low-substituted hydroxypropyl cellulose (L-HPC), hydroxyethylmethyl cellulose (HEMC), carboxypolymethylene (Carbomer), methylcellulose (MC), ethylcellulose (EC), hydroxyethyl cellulose (HEC), celluloseacetate, polyvinylpyrrolidone(PVP), crosslinked polyvinylpyrrolidone (Crospovidone), micronized crosslinked polyvinylpyrrolidone (crospovidone micronized), carboxymethylcellulose sodium (croscarmellose sodium, CMC Na), carboxymethylcellulose calcium (croscarmellose calcium, CMC Ca), crosslinked carboxymethylcellulose (Crosslinked CMC), microcrystalline cellulose (MCC), silicified microcrystalline cellulose (silicified MCC), cellulose powder, carboxymethyl starch (sodium starch glycolate), starch (maize starch, potato starch, rize starch, wheat starch, tapioca starch), pregelatinized starch or a combination thereof in an amount of particularly 40% by weight or more of the total weight of the core or dal layer and b) atorvastatin.

The invention provides a physically stable composition comprising a) a core or dal layer comprising at least S-[2-([[1-(2-ethylbutyl)-cyclohexyl]-carbonyl]amino)phenyl]2-methylpropanethioate, a hydrophobic and water instable cholesteryl ester transfer protein (CETP) inhibitor embedded in a chemically protective hygroscopic polymer matrix tablet consisting of at least one hygroscopic polymer e.g. hydroxypropylmethyl cellulose (HPMC), hydroxypropyl cellulose (HPC), low-substituted hydroxypropyl cellulose (L-HPC), hydroxyethylmethyl cellulose (HEMC), carboxypolymethylene (Carbomer), methylcellulose (MC), ethylcellulose (EC), hydroxyethyl cellulose (HEC), celluloseacetate, polyvinylpyrrolidone (PVP), crosslinked polyvinylpyrrolidone (Crospovidone), micronized crosslinked polyvinylpyrrolidone (crospovidone micronized), carboxymethylcellulose sodium (croscarmellose sodium, CMC Na), carboxymethylcellulose calcium (croscarmellose calcium, CMC Ca), crosslinked carboxymethylcellulose (Crosslinked CMC), microcrystalline cellulose (MCC), silicified microcrystalline cellulose (silicified MCC), cellulose powder, carboxymethyl starch (sodium starch glycolate), starch (maize starch, potato starch, rize starch, wheat starch, tapioca starch), pregelatinized starch or a combination thereof in an amount of particularly 40% by weight or more of the total weight of the core or dal layer; and b) a second or out layer comprising atorvastatin.

In particular, the present invention provides a physically stable composition comprising:

a) at least S-[2-([[1-(2-ethylbutyl)-cyclohexyl]-carbonyl]amino)phenyl]2-methylpropanethioate embedded in a chemically protective hygroscopic polymer matrix tablet consisting of hydroxypropylmethyl cellulose, carboxymethylcellulose sodium, microcrystalline cellulose and micronized crosslinked polyvinylpyrrolidone; and

b) atorvastatin.

The invention provides a physically stable composition comprising a) at least S-[2-([[1-(2-ethylbutyl)-cyclohexyl]-carbonyl]amino)phenyl]2-methylpropanethioate embedded in a chemically protective hygroscopic polymer matrix tablet consisting of hydroxypropylmethyl cellulose, carboxymethylcellulose sodium, microcrystalline cellulose and micronized crosslinked polyvinylpyrrolidone in an amount of particularly 40% by weight or more of the total weight of the core or dal layer and b) atorvastatin.

Usually bringing a moisture sensitive active pharmaceutical ingredient in contact with a high amount of hygroscopic polymers such as HPMC, HPC, PVP, Crospovidone, CMC, crosslinked CMC and MC is considered critical to physical stability.

Surprisingly it was found that in case of S-[2-([[1-(2-ethylbutyl)-cyclohexyl]-carbonyl]amino)phenyl]2-methylpropanethioate, a hydrophobic hydrolysis sensitive CETP inhibitor, a converse effect could be observed. It was possible to stabilize both, the active pharmaceutical ingredient and the immediate release tablet by embedding the active in a hygroscopic polymeric matrix comprising of:

| | Relative amount of the total |
|---|---|
| | weight of the core or dal layer |
| Ingredient | [%] |
| S-[2-([[1-(2-ethylbutyl)-cyclohexyl]- | >50% |
| carbonyl]amino)phenyl]2-methyl- | |
| propanethioate | |
| Water insoluble, hygroscopic polymer | >40% |
| Water soluble, hygroscopic polymer | >4% |
| Other Excipients | <6% |

Furthermore, it was surprisingly found that increasing the amount of hydroscopic polymers from its usual range of 10 to 20% by weight to more than 30% by weight of the total weight of the core or dal layer in the presence of hydrophobic compound did not lead to capping or cracking of the immediate-release tablet composition as would have been expected. Therefore the hydrophobic compound prevents the formation of cracks of the immediate release tablet when more than 30% by weight of the total weight of the core or dal layer of the tablet consists of hygroscopic polymeric excipients.

In another embodiment, the present invention provides a composition comprising:
a) 48% to 55% by weight of the total weight of the core or dal layer of S-[2-([[1-(2-ethylbutyl)-cyclohexyl]-carbonyl]amino)phenyl]2-methylpropanethioate (a hydrophobic and water instable cholesteryl ester transfer protein (CETP) inhibitor); and
at least 30% by weight of the total weight of the core or dal layer of hygroscopic polymeric excipients by composition weight, particularly 44% to 50% by weight of the total weight of the core or dal layer; and
b) atorvastatin

In another embodiment, the present invention provides a composition comprising:
a) 48% to 55% by weight of the total weight of the core or dal layer of S-[2-([[1-(2-ethylbutyl)-cyclohexyl]-carbonyl]amino)phenyl]2-methylpropanethioate;
40% to 45% by weight of the total weight of the core or dal layer of water insoluble hygroscopic polymer; and
4% to 5% by weight of the total weight of the core or dal layer of water soluble hygroscopic polymer; and

b). atorvastatin.

In another embodiment, the present invention provides a composition comprising:
a) 48% to 55% by weight of the total weight of the core or dal layer of S-[2-([[1-(2-ethylbutyl)-cyclohexyl]-carbonyl]amino)phenyl]2-methylpropanethioate (a hydrophobic and water instable cholesteryl ester transfer protein (CETP) inhibitor); and
at least 30% by weight, particularly 44% to 50% by weight of the total weight of the core or dal layer of hydroxypropylmethyl cellulose, croscarmellose sodium, microcrystalline cellulose and micronized crosslinked polyvinylpyrrolidone; and
b) atorvastatin.

In another embodiment, the present invention provides a composition comprising:
a) 48% to 55% by weight o of the total weight of the core or dal layer f S-[2-([[1-(2-ethylbutyl)-cyclohexyl]-carbonyl]amino)phenyl]2-methylpropanethioate (a hydrophobic and water instable cholesteryl ester transfer protein (CETP) inhibitor);
4% to 8% by weight of the total weight of the core or dal layer of croscarmellose sodium; and
35% to 44% by weight of the total weight of the core or dal layer of hydroxypropylmethyl cellulose, microcrystalline cellulose and crospovidone micronized; and
b) atorvastatin.

In another embodiment, the present invention provides a composition comprising:
a) 48% to 55% by weight of the total weight of the core or dal layer of S-[2-([[1-(2-ethylbutyl)-cyclohexyl]-carbonyl]amino)phenyl]2-methylpropanethioate (a hydrophobic and water instable cholesteryl ester transfer protein (CETP) inhibitor);
less than 12% by weight of the total weight of the core or dal layer of crospovidone micronized; and
35% to 44% by weight of the total weight of the core or dal layer of hydroxypropylmethyl cellulose, microcrystalline cellulose and croscarmellose sodium; and
b) atorvastatin.

In another embodiment, the present invention provides a composition comprising:
a) S-[2-([[1-(2-ethylbutyl)-cyclohexyl]-carbonyl]amino)phenyl]2-methylpropanethioate;
croscarmellose sodium; and
b) atorvastatin.

In another embodiment, the present invention provides a composition comprising:
a) S-[2-([[1-(2-ethylbutyl)-cyclohexyl]-carbonyl]amino)phenyl]2-methylpropanethioate;
microcrystalline cellulose;
crospovidone micronized;
hydroxypropylmethyl cellulose;
croscarmellose Sodium; and
b) atorvastatin

In another embodiment, the present invention provides a composition comprising:
a) S-[2-([[1-(2-ethylbutyl)-cyclohexyl]-carbonyl]amino)phenyl]2-methylpropanethioate;
mannitol;
crospovidone micronized;
hydroxypropylmethyl cellulose;
croscarmellose Sodium; and
b) atorvastatin.

In another embodiment, the present invention provides a composition comprising:
a) S-[2-([[1-(2-ethylbutyl)-cyclohexyl]-carbonyl]amino)phenyl]2-methylpropanethioate;
mannitol;
crospovidone micronized;
hydroxypropylmethyl cellulose;
croscarmellose Sodium;
microcrystalline cellulose; and
b) atorvastatin.

In another embodiment the present invention provides a composition comprising:
a) 48% to 55% by weight of the total weight of the core or dal layer of S-[2-([[1-(2-ethylbutyl)-cyclohexyl]-carbonyl]amino)phenyl]2-ethylpropanethioate;
24% to 26% by weight of the total weight of the core or dal layer of microcrystalline cellulose;
11% to 12% by weight of the total weight of the core or dal layer of crospovidone micronized;
4% to 5% by weight of the total weight of the core or dal layer of hydroxypropylmethyl cellulose;
4% to 6% by weight of the total weight of the core or dal layer of croscarmellose sodium; and
b) atorvastatin.

In another embodiment the present invention provides a composition comprising:
a) 48% to 55% by weight of the total weight of the core or dal layer of S-[2-([[1-(2-ethylbutyl)-cyclohexyl]-carbonyl]amino)phenyl]2-ethylpropanethioate;
24% to 26% by weight of the total weight of the core or dal layer of microcrystalline cellulose;
11% to 12% by weight of the total weight of the core or dal layer of crospovidone micronized;
4% to 5% by weight of the total weight of the core or dal layer of hydroxypropylmethyl cellulose;
4% to 6% by weight of the total weight of the core or dal layer of croscarmellose sodium;
0 to 1% by weight of the total weight of the core or dal layer of magnesium stearate;
0 to 1% by weight of the total weight of the core or dal layer of colloidal silicon dioxide;
0 to 1% by weight of the total weight of the core or dal layer of sodium stearyl fumarate; and
b) atorvastatin.

In certain embodiment of the present invention, the active coating comprises:
3% to 55% by weight of the total weight of the active coating of atorvastatin;
10% to 50% by weight of the total weight of the active coating of a film forming polymer selected from polyvinyl alcohol, hydroxypropyl methylcellulose, hydroxyl propylcellulose, polyvinyl alcohol-poly ethylene glycol graft copolymer or copovidone (vinylpyrrolidone-vinylacetate copolymers) or a combination thereof;
0% to 50% by weight of the total weight of the active coating of a filler such as lactose monohydrate or microcrystalline cellulose;
0 to 5% by weight of the total weight of the active coating of a plasticizer such as triacetin, triethylcitrate or poly ethylene glycol;
0% to 45% by weight of the total weight of the active coating of an glidant/antisticking agent like talcum, glycerinmonostearate or others;
0% to 2% by weight of the total weight of the active coating of a thickener like Sodium carboxymethyl cellulose or hydroxyl ethylcellulose or others;
0% to 25% by weight of the total weight of the active coating of a color like titandioxide, iron oxides or any other colour or mixture thereof;

In certain embodiment of the present invention, the active coating comprises:
atorvastatin;
Polyvinyl alcohol;
croscarmellose sodium;
triacetin;
talcum; and
simethicone.

In certain embodiment of the present invention, the active coating comprises:
atorvastatin;
Polyvinyl alcohol;
croscarmellose sodium;
triacetin;
talcum; and
simethicone.

In certain embodiment of the present invention, the active coating comprises:
45% to 55% by weight of the total weight of the active coating of atorvastatin;
10% to 30% by weight of the total weight of the active coating of polyvinyl alcohol;
0% to 5% by weight of the total weight of the active coating of croscarmellose sodium;
0% to 1% by weight of the total weight of the active coating of Triacetin;
5% to 40%, more particularly 10% to 25%, most particularly between 18% to 22% by weight of the total weight of the active coating of talcum; and
3% to 8%, more particularly between 4.5% and 5.5% by weight of the total weight of the active coating of simethicone.

In certain embodiment of the invention, the composition comprises
a) S-[2-([[1-(2-ethylbutyl)-cyclohexyl]-carbonyl]amino)phenyl]2-methylpropanethioate;
microcrystalline cellulose;
crospovidone micronized;
hydroxypropylmethyl cellulose;
croscarmellose sodium; and
b) atorvastatin.;
Polyvinyl alcohol;
croscarmellose sodium;
triacetin;
calcum; and
simethicone.

In another embodiment according to the present invention, the active coating comprising atorvastatin comprises:
5.41, 10.82, 21.65 or 43.28 mg of atorvastatin;
0.6 to 4.8 mg (0.18 to 1.44 mg)¹ of Simethicone Suspension USP (30% solids);
20.00 to 60.00 mg of PVA EG-05 PW;
0.00 to 40.00 mg of Lactose monohydrate;
2.00 to 6.00 mg of Triacetin;
18.00 to 54.00 mg of Talcum; and
0.00 to 0.50 mg of Sodium Carboxymethyl cellulose.

In another embodiment according to the present invention, the atv layer comprising atorvastatin comprises:
atorvastatin;
Lactose;
Microcrystalline Cellulose;
Croscarmellose Sodium;
Magnesium carbonate, calcium carbonate or magnesium oxide;
hydroxypropyl cellulose (HPC);
Polysorbate 80; and
Magnesium stearate.

In certain embodiment of the present invention, the atv layer comprises:
1% to 18% by weight of the total weight of the atv layer of atorvastatin;
5% to 50% by weight of the total weight of the atv layer of lactose monohydrate,
10% to 55% by weight of the total weight of the atv layer of microcrystalline cellulose;
3 to 15% by weight of the total weight of the atv layer of croscarmellose sodium;
3% to 50% by weight of the total weight of the atv layer of a pharmaceutically acceptable stabilizing additive, in particular CaCO3 or MgCO3 or MgO;
0.3% to 5% by weight of the total weight of the atv layer of hydroxyl propylcellulose;
0 to 1% by weight of the total weight of the atv layer of polysorbate 80; and
0 to 1.5% by weight of the total weight of the atv layer of magnesium stearate.

In a particular embodiment, the composition herein is film coated, in particular with a polymer coating, such as HPMC and HPC or polyvinyl alcohol-polyethylene glycol (Kollicoat® IR) or a polyvinyl alcohol based coat (PVA-based coat), particularly with 30 mg or less PVA-based coat, more particularly with 20 mg PVA-based coat.

In a particular embodiment, the composition herein is film coated with vinylpyrrolidone-vinyl acetatecopolymer (PVP VA 64 also known as Kollidon® VA 64), Triethylcitrate, Talcum and Titan dioxide.

Alternatively to the film forming polymer, the plasticizer, the filler and colours additives a ready to use mixture like Opadry II clear can be used.

In certain embodiment of the present invention, the core comprising dalcetrapib is separated from the active coating comprising atorvastatin by a separation layer. In particular, the separation layer comprises polyvinyl alcohol, triacetin and talcum. In another embodiment the separation layer comprises vinylpyrrolidone-vinyl acetatecopolymer (PVP VA 64 also known as Kollidon® VA 64) Triacetin and Talcum.

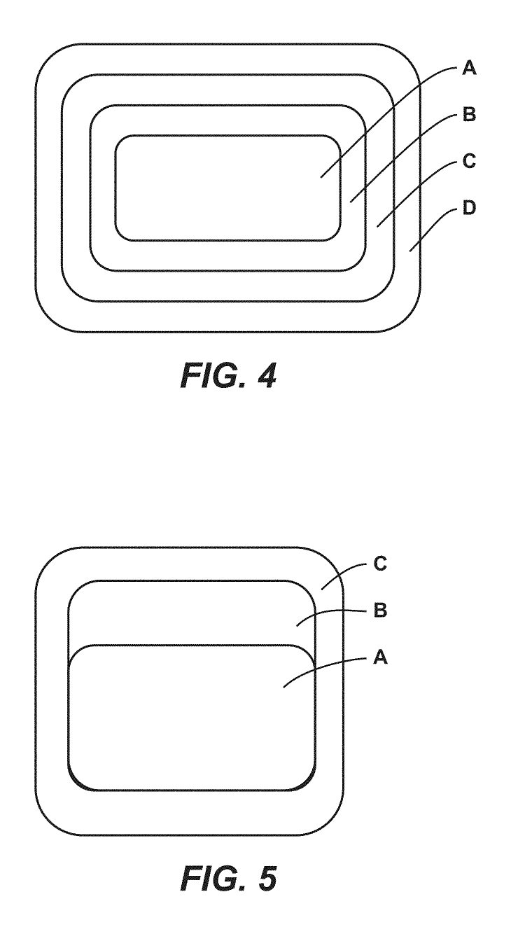

In certain embodiment of the present invention, the composition comprises a seal coat or a film coat as shown in FIG. 4. In particular, the seal coat comprises polyvinyl alcohol, triacetin, titan dioxide and talcum.

In certain embodiments of the present invention, the composition is a pharmaceutical composition.

The pharmaceutical composition can be, for example, in the form of a pill, capsule or tablet, each containing a predetermined amount of S-[2-([[1-(2-ethylbutyl)-cyclohexyl]-carbonyl]amino)phenyl]2-methylpropanethioate and atorvastatin and in particular coated for ease of swallowing, in the form of a powder or granules. In particular, the pharmaceutical composition is in the form of a tablet comprising S-[2-([[1-(2-ethylbutyl)-cyclohexyl]-carbonyl]amino)phenyl]2-methylpropanethioate, atorvastatin and the components of the tablet utilized and described therein. For oral administration, fine powders or granules may contain diluting, dispersing and/or surface active agents and may be present, for example, in capsules or sachets in the dry state, or in tablets wherein binders and lubricants may be included. Components such as sweeteners, flavoring agents, preservatives, suspending agents, thickening agents, and/or emulsifying agents also may be present in the pharmaceutical composition.

In certain embodiments of the present invention, the composition comprises 100 mg to 600 mg of S-[2-([[1-(2-ethylbutyl)-cyclohexyl]-carbonyl]amino)phenyl]2-methylpropanethioate. In particular, the composition comprises 150 mg to 450 mg of S-[2-([[1-(2-ethylbutyl)-cyclohexyl]-carbonyl]amino)phenyl]2-methylpropanethioate. More particularly, the composition comprises 250 mg to 350 mg of S-[2-([[1-(2-ethylbutyl)-cyclohexyl]-carbonyl]amino)phenyl]2-methylpropanethioate. Most particularly, the composition comprises 250 mg to 350 mg of S-[2-([[1-(2-ethylbutyl)-cyclohexyl]-carbonyl]amino)phenyl]2-methylpropanethioate.

In certain embodiments of the present invention, the composition comprises 300 mg of S-[2-([[1-(2-ethylbutyl)-cyclohexyl]-carbonyl]amino)phenyl]2-methylpropanethioate and 5 mg, 10 mg, 20 mg or 40 mg of atorvastatin.

In another embodiment of the present invention, the composition comprises for pediatric use 25 mg to 300 mg of S-[2-([[1-(2-ethylbutyl)-cyclohexyl]-carbonyl]amino)phenyl]2-methylpropanethioate. In particular the pediatric composition comprises 75 mg to 150 mg of S-[2-([[1-(2-ethylbutyl)-cyclohexyl]-carbonyl]amino)phenyl]2-methylpropanethioate.

In another embodiment of the present invention, the composition comprises for pediatric use 150 mg of S-[2-([[1-(2-ethylbutyl)-cyclohexyl]-carbonyl]amino)phenyl]2-methylpropanethioate and 5 mg, 10 mg or 20 mg of atorvastatin.

The CETP inhibitor can be administered to the mammal at any suitable dosage (e. g., to achieve a therapeutically effective amount). For example, a suitable dose of a therapeutically effective amount of Compound I for administration to a patient will be between approximately 100 mg to about 1800 mg per day. A desirable dose is particularly about 300 mg to about 900 mg per day. A preferred dose is about 600 mg per day.

In another embodiment the invention provides a kit comprising a composition comprising a therapeutically effective amount of S-[2-([[1-(2-ethylbutyl)-cyclohexyl]-carbonyl]amino)phenyl]2-methylpropanethioate and atorvastatin and at least 30% by weight of hygroscopic polymeric excipients by composition weight, prescribing information also known as “leaflet”, a blister package or bottle (HDPE or glass) and a container. The prescribing information particularly includes the advice to a patient regarding the administration of S-[2-([[1-(2-ethylbutyl)-cyclohexyl]-carbonyl]amino)phenyl]2-methylpropanethioate with food, especially to improve the bioavailability of S-[2-([[1-(2-ethylbutyl)-cyclohexyl]-carbonyl]amino)phenyl]2-methylpropanethioate. In more particular, the prescribing information includes the advice to a patient regarding the administration of S-[2-([[1-(2-ethylbutyl)-cyclohexyl]-carbonyl]amino)phenyl]2-methylpropanethioate and atorvastatin with food, especially to improve the bioavailability of S[2-([[1-(2-ethylbutyl)-cyclohexyl]-carbonyl]amino)phenyl]2-methylpropanethioate.

In another embodiment the invention provides a kit comprising a composition as described herein, prescribing information also known as “leaflet”, a blister package or bottle (HDPE or glass) and a container. The prescribing information particularly includes the advice to a patient regarding the administration of the S-[2-([[1-(2-ethylbutyl)-cyclohexyl]-carbonyl]amino)phenyl]2-methylpropanethioate with food, especially to improve the bioavailability of the S-[2-([[1-(2-ethylbutyl)-cyclohexyl]-carbonyl]amino)phenyl]2-methylpropanethioate. More particularly the prescribing information includes the advice to a patient regarding the administration of the S-[2-([[1-(2-ethylbutyl)-cyclohexyl]-carbonyl]amino)phenyl]2-methylpropanethioate and atorvastatin with food, especially to improve the bioavailability of the S-[2-([[1-(2-ethylbutyl)-cyclohexyl]-carbonyl]amino)phenyl]2-methylpropanethioate.

In another embodiment, the invention provides a kit comprising a composition comprising a therapeutically effective amount of S-[2-([[1-(2-ethylbutyl)-cyclohexyl]-carbonyl]amino)phenyl]2-methylpropanethioate and atorvastatin, and at least 30% by weight of hygroscopic polymeric excipients by composition weight, prescribing information, a blister package or bottle and a container. In particular embodiment the invention provides the kit as described herein, wherein the prescribing information includes the advice to a patient regarding the administration of S-[2-([[1-(2-ethylbutyl)-cyclohexyl]-carbonyl]amino)phenyl]2-methylpropanethioate with food.

In another embodiment, the invention provides a tablet comprising the composition as herein described.

In another embodiment, the invention provides a composition as herein described for preparing a medicament for the treatment or prevention of cardiovascular disorder, in particular wherein the S-[2-([[1-(2-ethylbutyl)-cyclohexyl]-carbonyl]amino)phenyl]2-methylpropanethioate is administered at a daily dose of 100 mg to 1800 mg, particularly 300 mg to 900 mg, more particularly 600 mg, more particularly wherein S-[2-([[1-(2-ethylbutyl)-cyclohexyl]-carbonyl]amino)phenyl]2-methylpropanethioate is administered with food.

In another embodiment, the invention provides a process for the preparation of the composition comprising the following steps:
a) Mixing and granulating, S-[2-([[1-(2-ethylbutyl)-cyclohexyl]-carbonyl]amino)phenyl]2-methylpropanethioate, crospovidone, microcrystalline cellulose, croscarmellose sodium and hydroxypropyl methylcellulose;
b) Spraying up to 0.5% by weight of HPMC in water or in 10%-30% ethanol by weight/70%-90% water by weight, onto the granulates obtained according to step a);
c) drying the granulates;
d) blending microcrystalline Cellulose, colloidal silicon dioxide and sodium stearylfumarate with the dry granulates obtained according to step c);
e) compressing the tablets;
f) aqueous film coating with the separation layer, in particular the separation layer comprises polyvinyl alcohol, triacetin and talcum;
g) aqueous film coating with the active coating, in particular the active coating comprises atorvastatin, polyvinyl alcohol, triacetin, talcum, simethicone and optionally croscarmellose sodium.

In another embodiment, the invention provides a process for the preparation of the composition comprising the following steps:
a) Mixing and granulating, S-[2-([[1-(2-ethylbutyl)-cyclohexyl]-carbonyl]amino)phenyl]2-methylpropanethioate, crospovidone, microcrystalline cellulose, croscarmellose sodium and hydroxypropyl methylcellulose;
b) Spraying up to 0.5% by weight of HPMC in water or in 10%-30% ethanol by weight/70%-90% water by weight, onto the granulates obtained according to step a);
c) drying the granulates;
d) blending microcrystalline Cellulose, colloidal silicon dioxide and sodium stearylfumarate with the dry granulates obtained according to step c);
e) compressing the tablets;
f) film coating with the separation layer, in particular the separation layer comprises polyvinyl alcohol, triacetin and talcum;
g) aqueous film coating with the active coating, in particular the active coating comprises atorvastatin, polyvinyl alcohol, triacetin, talcum, and optionally simethicone and/or optionally croscarmellose sodium; and
h) aqueous film coating with a seal coating, in particular the seal coating comprises polyvinyl alcohol, triacetin and talcum, optionally titan dioxide and/or Colouring agent;

In another embodiment the present invention provides a process for the preparation of the composition as described herein, which comprises the following steps:
a) Mixing and granulating, S-[2-([[1-(2-ethylbutyl)-cyclohexyl]-carbonyl]amino)phenyl]2-methylpropanethioate, Crospovidone micronized, microcrystalline Cellulose, Croscarmellose sodium and optionally with Hydroxypropylmethyl cellulose;
b) Spraying up to 0.5% by weight of Hydroxypropylmethyl cellulose in water or in 10%-30% ethanol by weight/70%-90% water by weight, more particularly in 20% Ethanol by weight/80% water by weight onto the granulates obtained according to step a);
c) drying the granulates;
d) blending microcrystalline Cellulose, colloidal silicon dioxide and sodium stearylfumarate with the dry granulates obtained according to step c);
e) compressing the tablets;
f) film coating with the separation layer, in particular the separation layer comprises polyvinyl alcohol, triacetin and talcum;
g) aqueous film coating with the active coating, in particular the active coating comprises atorvastatin, polyvinyl alcohol, croscarmellose sodium, triacetin, talcum and optionally simethicone and/or croscarmellose sodium;
h) aqueous film coating with a seal coating, in particular the seal coating comprises polyvinyl alcohol, triacetin and talcum, optionally titan dioxide and/or Colouring agent;

In another embodiment the present invention provides a process for the preparation of the composition as described herein, which comprises the following steps:
a) Mixing and granulating, S-[2-([[1-(2-ethylbutyl)-cyclohexyl]-carbonyl]amino)phenyl]2-methylpropanethioate, crospovidone micronized, mannitol, croscarmellose sodium and Hydroxypropylmethyl cellulose;
b) spraying up to 0.5% by weight of hydroxypropylmethyl cellulose in water or in 10%-30% ethanol by weight/70%-90% water by weight, more particularly in 20% Ethanol by weight/80% water by weight onto the granulates obtained according to step a);
c) drying the granulates; and
d) blending microcrystalline Cellulose, colloidal silicon dioxide and sodium stearylfumarate with the dry granulates obtained according to step c);
e) compressing the tablets;
f) film coating with the separation layer, in particular the separation layer comprises polyvinyl alcohol, triacetin and talcum;
g) aqueous film coating with the active coating, in particular the active coating comprises atorvastatin, polyvinyl alcohol, croscarmellose sodium, triacetin, talcum and optionally simethicone and/or croscarmellose sodium;
h) aqueous film coating with a seal coating, in particular the seal coating comprises polyvinyl alcohol, triacetin and talcum, optionally titan dioxide and/or Colouring agent.

In another embodiment the present invention provides a process for the preparation of the composition as described herein, which comprises the following steps:
a) Mixing and granulating, the water instable compound with a waxy consistency, crospovidone micronized, microcrystalline Cellulose and Croscarmellose sodium;
b) spraying the hydroxypropylmethyl cellulose in 10%-30% ethanol by weight/70%-90% water by weight, more particularly in 20% Ethanol by weight/80% water by weight onto the granulates obtained according to step a);
c) drying the granulates;
d) blending microcrystalline Cellulose, colloidal silicon dioxide and sodium stearylfumarate with the dry granulates obtained according to step c);
e) compressing the tablets;
f) film coating with the separation layer, in particular the separation layer comprises polyvinyl alcohol, triacetin and talcum;
g) aqueous film coating with the active coating, in particular the active coating comprises atorvastatin, polyvinyl alcohol, croscarmellose sodium, triacetin, talcum and optionally simethicone and/or croscarmellose sodium;
h) aqueous film coating with a seal coating, in particular the seal coating comprises polyvinyl alcohol, triacetin and talcum, optionally titan dioxide and/or Colouring agent.

In another embodiment, the invention provides a process for the preparation the dal layer comprising the following steps:
a) Mixing and granulating, S-[2-([[1-(2-ethylbutyl)-cyclohexyl]-carbonyl]amino)phenyl]2-methylpropanethioate, crospovidone, microcrystalline cellulose, croscarmellose sodium and hydroxypropyl methylcellulose;
b) Spraying up to 0.5% by weight of HPMC in water or in 10%-30% ethanol by weight/70%-90% water by weight, onto the granulates obtained according to step a);
c) drying the granulates;
d) blending microcrystalline Cellulose, colloidal silicon dioxide and sodium stearylfumarate with the dry granulates obtained according to step c);

In another embodiment, the invention provides a process for the preparation the atv layer comprising the following steps:
a) Sieving each of the following components, [R-(R*,R*)]-2-(4-fluorophenyl)-β,δ-dihydroxy-5-(1-methylethyl)-3-phenyl-4-[(phenylamino)carbonyl]-1H-pyrrole-1-heptanoic acid calcium salt (2:1) trihydrate, lactose, microcrystalline cellulose, calcium carbonate, magnesium carbonate or magnesium oxide, and croscarmellose sodium and mixing them together to obtain a dry powder blend;
b) Granulating in a high shear granulator by spraying HPC and Polysorbate 80 in water, onto dry powder blend obtained according to step a);
c) wet sieving of the obtained granules and drying the granulates;
d) dry sieving of the granules through a 0.9 mm screen and blending microcrystalline Cellulose (for the 5 mg and 10 mg dose strength of atorvastatin) , croscarmellose sodium, lactose (for the 5 mg and 10 mg dose strength of atorvastatin) and magnesium stearate with the dry granulates obtained according to step c);

In another embodiment, the invention provides a process for the preparation the composition according to the present invention comprising the following steps:
a) compressing the blend obtained from dal layer step d) and the blend obtained from atv layer step d); and
b) film coating the compressed tablet obtain in step a)

### Manufacturing Process:

Herein, the API 1 refers to the active substance S-[2-([[1-(2-ethylbutyl)-cyclohexyl]-carbonyl]amino)phenyl]2-methylpropanethioate which is a hydrophobic, water instable compound with a waxy consistency. The API 2 refers in examples 2 to 70 to the active substance [R-(R*,R*)]-2-(4-fluorophenyl)-β,δ-dihydroxy-5-(1-methylethyl)-3-phenyl-4-[(phenylamino)carbonyl]-1H-pyrrole-1-heptanoic acid, calcium salt (2:1) trihydrate. In the examples 1 to 70 the API 1 refers to S-[2-([[1-(2-ethylbutyl)-cyclohexyl]-carbonyl]amino)phenyl]2-methylpropanethioate of formula (I′) in crystalline form.

The composition of the present invention may be prepared according to any known process which results in keeping the API 1 substantially in crystalline form (the amount of the hydrophobic, API 1 in amorphous does not exceed 10% by weight). Furthermore, the composition of the present invention may be prepared according to any known process which results in keeping the API 1 substantially in crystalline form (the amount of the hydrophobic, water instable compound with a waxy consistency substantially in amorphous does not exceed 10% by weight).

The composition of the present invention may be prepared according to any known process which results in keeping the API 2 substantially in crystalline form (the amount of API 2 in amorphous does not exceed 10% by weight). Furthermore, the composition of the present invention may be prepared according to any known process which results in keeping the API 2 substantially in crystalline form (the amount of API 2 in amorphous does not exceed 10% by weight).

The process for preparing the core or dal layer composition according to the invention may comprise the following steps:
1) dissolving Hydroxypropylmethyl cellulose (0.5% by weight of total Hydroxypropylmethyl cellulose) in 20% Ethanol by weight and 80% water by weight under constant stirring;
2) loading granulator (high shear mixer: e.g. Diosna®) vertical granulator with bottom driven impeller, with S-[2-([[1-(2-ethylbutyl)-cyclohexyl]-carbonyl]amino)phenyl]2-methylpropanethioate, Crospovidone micronized, microcrystalline Cellulose, Croscarmellose sodium and remainder of Hydroxypropylmethyl cellulose;
3) mixing the dry granulate components using impeller and chopper;
4) humidifying the granulate by spraying of the granulation fluid under constant mixing using impeller and chopper;
5) kneading the wetted granulate using impeller and chopper;
6) discharging the wet granulate, screening it over a conical mill [e.g. Frewitt® (screening mill with rotating impeller)); fitted with a 10 mm square screen and loading it into the fluid bed dryer
7) drying the granulate in the fluid bed dryer (e.g Glatt®) with inlet air temperature≦60° C. until the final LOD (loss on drying) of ≦3.5% by weight is reached;
8) discharging the dried granulate and milling it using an impact mill fitted with a 1.5 mm round perforation screen (e.g. Frewitt® (impact mill with rotating hammer);
9) adding the components of the external phase (e.g. microcrystalline Cellulose, colloidal silicon dioxide and sodium stearylfumarate) over a sieve fitted with a 1 mm round perforation screen to the granulate
10) blending all the components in a bin blender (e.g. Tumblemix® (tumble blending in bin blender));
11) compressing the tablets on a rotary tablet press with low compression force (around 6 kN), (e.g. Korsch® (power assisted);

The process for preparing active coating composition according to the invention may comprise the following steps:

The process for preparing by layer tablet composition according to the invention may comprise the following steps:
12) preparing the coating suspension for the separation layer by dispersing the polyvinyl alcohol in a part of the water;
13) dispersing the talcum in another part of the water by homogeneization
14) preparing a mixture of PVA dissolved in water as obtained under 12), talcum suspended in water as obtained under 13), triacetin and any remaining water;
15) loading the tablets obtained under 11) into the perforated coating drum;
16) preheating the tablets in the perforated drum until an exhaust temperature of 40 to 45° C. is reached;
17) spraying the coating suspension of the separation layer obtained under 14) onto the tablets under constant rotation of the perforated coating drum;
18) drying the film coated tablets under constant rotation of the perforated coating drum;
19) preparing the coating suspension for the active coating layer by dispersing the polyvinyl alcohol in a part of the water;
20) dispersing simethicone, atorvastatin and talcum in another part of the water by homogeneization
21) preparing a mixture of PVA dissolved in water as obtained under 19), the suspension containing atorvastatin as obtained under 20), triacetin and any remaining water;
22) spraying the coating suspension of the active coating layer obtained under 21) onto the tablets under constant rotation of the perforated coating drum;
23) drying the film coated tablets under constant rotation of the perforated coating drum;
24) preparing the coating suspension for the seal coat by dispersing the polyvinyl alcohol in a part of the water;
25) dispersing talcum, titan dioxide and/or any color in another part of the water by homogeneization
26) preparing a mixture of PVA dissolved in water as obtained under 24), the suspension as obtained under 25), triacetin and any remaining water;
27) spraying the coating suspension of the seal coat obtained under 26) onto the tablets under constant rotation of the perforated coating drum;
28) drying the film coated tablets under constant rotation of the perforated coating drum;
29) discharging the film coated tablets;
30) imprinting of the film coated tablets

Other features and embodiments of the invention will become apparent from the following examples which are given for illustration of the invention rather than for limiting its intended scope.

Examples 1 to 46 and placebo example A were prepared according to the above mentioned general processes, wherein the API 1 for example A has been replaced with mannitol. Examples 47 to 70 are prepared according to the above mentioned general processes. Examples 22 to 30, 36 to 46 and 62 to 70 were all film coated with 20 mg PVA-based coat (e.g. Opadry® e.g. Opadry II white 85F18422).

### Example No 1

| | | | | hygroscopic | |
|---|---|---|---|---|---|
| | | | | polymer | |
| | | Mass/Unit | Amount/Unit | Water | Water |
| | Ingredient | [mg] | [%] | insoluble | soluble |
| Core or | API 1 | 300.00 | 52.54 | N/A | N/A |
| dal | Microcrystalline Cellulose (Type 101) | 66.00 | 11.56 | + | |
| layer | Crospovidone micronized | 66.00 | 11.56 | + | |
| | HPMC (2910 3cp) | 24.00 | 4.20 | | + |
| | CMC Na | 34.00 | 5.95 | + | |
| | Colloidal Silicon Dioxide | 3.00 | 0.53 | N/A | N/A |
| | Sodium stearyfumarate | 3.00 | 0.53 | N/A | N/A |
| | Microcrystalline Cellulose (Type 102) | 75.00 | 13.35 | + | |
| | Total core tablet | 571.00 | 100.00 | | |
| | Amount hygroscopic polymers | | | 42.21% | 4.20% |
| | Total amount hygroscopic polymers | | | 46.41% | |

### Example No 2

| | | Mass/Unit |
|---|---|---|
| | Ingredient | [mg] |
| Core | According to example 1 | 571.00 |
| Separation | HPMC (2910 3cp) | 5.00 |
| Layer | HPC (Klucel LF) | 5.00 |
| | triacetin | 1.00 |
| | talcum | 9.00 |
| Active | API 2 | 5.41 |
| Coating | Triethylcitrate | 0.25 |
| | HPMC (2910 3cp) | 1.25 |
| | HPC (Klucel LF) | 1.25 |
| | Talcum | 2.25 |
| | Simethicone Suspension USP (30% solids) | 0.60 (0.18)¹ |
| Seal | HPMC (2910 3 cp) | 5.00 |
| Coat | HPC (Klucel LF) | 5.00 |
| | triethylcitrate | 1.00 |
| | Talcum | 4.50 |
| | Titan dioxide | 4.50 |

¹In suspension (solvent free)

### Example No 3

| | | Mass/Unit |
|---|---|---|
| | Ingredient | [mg] |
| Core | According to example 1 | 571.00 |
| Separation | HPMC (2910 3cp) | 5.00 |
| Layer | HPC (Klucel LF) | 5.00 |
| | triacetin | 1.00 |
| | talcum | 9.00 |
| Active | API 2 | 43.30 |
| Coating | Triethylcitrate | 2.00 |
| | HPMC (2910 3cp) | 10.00 |
| | HPC (Klucel LF) | 10.00 |
| | Talcum | 18.00 |
| | Simethicone Suspension USP (30% solids) | 4.80 (1.44)¹ |
| Seal | HPMC (2910 3cp) | 5.00 |
| Coat | HPC (Klucel LF) | 5.00 |
| | triethylcitrate | 1.00 |
| | Talcum | 4.50 |
| | Titan dioxide | 4.50 |

¹In suspension (solvent free)

### Example No 4

| | | Mass/Unit |
|---|---|---|
| | Ingredient | [mg] |
| Core | According to example 1 | 571.00 |
| Separation | Kollidon ® VA 64 | 10.00 |
| Layer | Triacetin | 1.00 |
| | Talcum | 9.00 |
| Active | API 2 | 5.41 |
| Coating | Simethicone Suspension USP (30% solids) | 0.60 (0.18)¹ |
| | Kollidon ® VA 64 | 2.50 |
| | Triethylcitrate | 0.25 |
| | Talcum | 2.25 |
| Seal | Kollidon ® VA 64 | 10.00 |
| Coat | Triethylcitrate | 1.00 |
| | Talcum | 4.50 |
| | Titan dioxide | 4.50 |

¹In suspension (solvent free)

### Example No 5

| | | Mass/Unit |
|---|---|---|
| | Ingredient | [mg] |
| Core | According to example 1 | 571.00 |
| Separation | Kollidon ® VA 64 | 10.00 |
| Layer | Triacetin | 1.00 |
| | Talcum | 9.00 |
| Active | API 2 | 43.30 |
| Coating | Simethicone Suspension USP (30% solids) | 4.80 (1.44)¹ |
| | Kollidon ® VA 64 | 20.00 |
| | Triethylcitrate | 2.00 |
| | Talcum | 18.00 |
| Se | Kollidon ® VA 64 | 10.00 |
| | Triethylcitrate | 1.00 |
| | Talcum | 4.50 |
| | Titan dioxide | 4.50 |

¹In suspension (solvent free)

### Example No 6

| | | Mass/Unit |
|---|---|---|
| | Ingredient | [mg] |
| Core | According to example 1 | 571.00 |
| Separation | Kollicoat IR | 10.50 |
| Layer | Talcum | 9.50 |
| Active | API 2 | 5.41 |
| Coating | Simethicone Suspension USP (30% solids) | 0.60 (0.18)¹ |
| | Kollicoat IR | 2.63 |
| | Talcum | 2.38 |
| Seal | Kollicoat IR | 10.50 |
| Coat | Talcum | 4.75 |
| | Titan dioxide | 4.75 |

¹In suspension (solvent free)

### Example No 7

| | | Mass/Unit |
|---|---|---|
| | Ingredient | [mg] |
| Core | According to example 1 | 571.00 |
| Separat | Kollicoat IR | 10.50 |
| | Talcum | 9.50 |
| Active | API 2 | 43.30 |
| Coating | Simethicone Suspension USP (30% solids) | 4.80 (1.44)¹ |
| | Kollicoat IR | 21.00 |
| | Talcum | 19.00 |
| Seal | Kollicoat IR | 10.50 |
| Coat | Talcum | 4.75 |
| | Titan dioxide | 4.75 |

¹In suspension (solvent free)

### Example No 8

| | | Mass/Unit |
|---|---|---|
| | Ingredient | [mg] |
| Core | According to example 1 | 571.00 |
| Separation | HPMC (2910 3cp) | 5.00 |
| Layer | HPC (Klucel LF) | 5.00 |
| | triacetin | 1.00 |
| | talcum | 9.00 |
| Active | API 2 | 5.41 |
| Coating | Triethylcitrate | 0.25 |
| | HPMC (2910 3cp) | 1.25 |
| | HPC (Klucel LF) | 1.25 |
| | Talcum | 2.25 |
| | Simethicone Suspension USP (30% solids) | 0.60 (0.18)¹ |
| Seal | HPMC (2910 3cp) | 5.00 |
| Coat | HPC (Klucel LF) | 5.00 |
| | triethylcitrate | 1.00 |
| | Talcum | 4.50 |
| | Titan dioxide | 4.50 |

¹In suspension (solvent free)

### Example No 9

| | | Mass/Unit |
|---|---|---|
| | Ingredient | [mg] |
| Core | According to example 1 | 571.00 |
| Separation | polyvinyl alcohol (PVA EG -05 PW) | 10.00 |
| Layer | triacetin | 1.00 |
| | talcum | 9.00 |
| Active | API 2 | 5.41 |
| Coating | polyvinyl alcohol (PVA EG -05 PW) | 2.50 |
| | CMC Na | 0.20 |
| | Triacetin | 0.25 |
| | Talcum | 2.25 |
| | Simethicone Suspension USP (30% solids) | 0.60 (0.18)¹ |
| Seal | polyvinyl alcohol (PVA EG -05 PW) | 10.00 |
| Coat | Triacetin | 1.00 |
| | Talcum | 4.50 |
| | Titan dioxide | 4.50 |

¹In suspension (solvent free)

### Example No 10

| | | Mass/Unit |
|---|---|---|
| | Ingredient | [mg] |
| Core | According to example 1 | 571.00 |
| Separation | polyvinyl alcohol (PVA EG -05 PW) | 10.00 |
| Layer | triacetin | 1.00 |
| | talcum | 9.00 |
| Active | API 2 | 43.30 |
| Coating | polyvinyl alcohol (PVA EG -05 PW) | 20.00 |
| | CMC Na | — |
| | Triacetin | 2.00 |
| | Talcum | 18.00 |
| | Simethicone Suspension USP (30% solids) | 4.80 (1.44)¹ |
| Seal | polyvinyl alcohol (PVA EG -05 PW) | 10.00 |
| Coat | Triacetin | 1.00 |
| | Talcum | 4.50 |
| | Titan dioxide | 4.50 |

¹In suspension (solvent free)

### Example No 11

| | | Mass/Unit |
|---|---|---|
| | Ingredient | [mg] |
| Core | According to example 1 | 571.00 |
| Separation | Opadry II white 85F18422 | 20.00 |
| Layer | | |
| Active | API 2 | 5.40 |
| Coating | Opadry II clear 85F29116 | 19.95 |
| | Calcium carbonate | 16.2 |
| | Simethicone Suspension USP (30% solids) | 0.45 (0.14)¹ |
| Seal | Opadry II brown 85F26792 | 15.00 |
| Coat | | |

¹In suspension (solvent free)

### Example No 12

| | | Mass/Unit |
|---|---|---|
| | Ingredient | [mg] |
| Core | According to example 1 | 571.00 |
| Separation | Opadry II white 85F18422 | 20.00 |
| Layer | | |
| Active | API 2 | 10.8 |
| Coating | Opadry II clear 85F29116 | 39.9 |
| | Calcium carbonate | 32.4 |
| | Simethicone Suspension USP (30% solids) | 0.9 (0.28)¹ |
| Seal | Opadry II brown 85F26792 | 15.00 |
| Coat | | |

¹In suspension (solvent free)

### Example No 13

| | | Mass/Unit |
|---|---|---|
| | Ingredient | [mg] |
| Core | According to example 1 | 571.00 |
| Separation | Opadry II white 85F18422 | 20.00 |
| Layer | | |
| Active Coating | API 2 | 21.6 |
| | Opadry II clear 85F29116 | 79.8 |
| | Calcium carbonate | 64.8 |
| | Simethicone Suspension USP (30% solids) | 1.8 (0.56)¹ |
| Seal | Opadry II brown 85F26792 | 15.00 |
| Coat | | |

¹In suspension (solvent free)

### Example No 14

| | | Mass/Unit |
|---|---|---|
| | Ingredient | [mg] |
| Core | According to example 1 | 571.00 |
| Separation | polyvinyl alcohol (PVA EG-05 PW) | 10.00 |
| Layer | Triacetin | 1.00 |
| | Talcum | 9.00 |
| Active Coating | API 2 | 5.41 |
| | polyvinyl alcohol (PVA EG-05 PW) | 2.50 |
| | Triacetin | 0.25 |
| | Talcum | 2.25 |
| | Simethicone Suspension USP (30% solids) | 0.6 (0.18)¹ |
| Seal Coat | polyvinyl alcohol (PVA EG-05 PW) | 10.00 |
| | Triacetin | 1.00 |
| | Talcum | 4.50 |
| | Titan dioxide | 4.50 |

¹In suspension (solvent free)

### Example No 15

| | | Mass/Unit |
|---|---|---|
| | Ingredient | [mg] |
| Core | According to example 1 | 571.00 |
| Separation | polyvinyl alcohol (PVA EG-05 PW) | 10.00 |
| Layer | Triacetin | 1.00 |
| | Talcum | 9.00 |
| Active Coating | API 2 | 5.41 |
| | polyvinyl alcohol (PVA EG-05 PW) | 20.00 |
| | Triacetin | 2.00 |
| | Talcum | 18.00 |
| | Simethicone Suspension USP (30% solids) | 4.8 (1.44)¹ |
| Seal | polyvinyl alcohol (PVA EG-05 PW) | 10.00 |
| Coat | Triacetin | 1.00 |
| | Talcum | 4.50 |
| | Titan dioxide | 4.50 |

¹In suspension (solvent free)

### Example No 16

| | | Mass/Unit |
|---|---|---|
| | Ingredient | [mg] |
| Dal | According to example 1 | 571.00 |
| layer | | |
| Atv Layer | API 2 | 5.41 |
| | Lactose Pulvis | 26.09 |
| | Microcrystalline Cellulose (PH 102) | 28.6 |
| | Croscarmellose Sodium | 2.18 |
| | Magnesium oxide | 15 |
| | HPC (Klucel LF) | 1.45 |
| | Polysorbate 80 (Tween 80) | 0.29 |
| | Lactose (Tablettose 70) | 102.34 |
| | Microcrystalline Cellulose (PH 102) | 128.16 |
| | Croscarmellose Sodium | 15.26 |
| | Magnesium stearate | 2.59 |

### Example No 17

| | | Mass/Unit |
|---|---|---|
| | Ingredient | [mg] |
| Dal | According to example 1 | 571.00 |
| layer | | |
| Atv Layer | API 2 | 10.82 |
| | Lactose Pulvis | 52.18 |
| | Microcrystalline Cellulose (PH 102) | 57.19 |
| | Croscarmellose Sodium | 4.35 |
| | Magnesium oxide | 30 |
| | HPC (Klucel LF) | 2.9 |
| | Polysorbate 80 (Tween 80) | 0.58 |
| | Lactose (Tablettose 70) | 86.77 |
| | Microcrystalline Cellulose (PH 102) | 66.9 |
| | Croscarmellose Sodium | 13.08 |
| | Magnesium stearate | 2.59 |

### Example No 18

| | | Mass/Unit |
|---|---|---|
| | Ingredient | [mg] |
| Dal | According to example 1 | 571.00 |
| layer | | |
| Atv Layer | API 2 | 21.64 |
| | Lactose Pulvis | 104.36 |
| | Microcrystalline Cellulose (PH 102) | 114.38 |
| | Croscarmellose Sodium | 8.7 |
| | Magnesium oxide | 60.00 |
| | HPC (Klucel LF) | 5.8 |
| | Polysorbate 80 (Tween 80) | 1.16 |
| | Croscarmellose Sodium | 8.73 |
| | Magnesium stearate | 1.61 |

### Example No 19

| | | Mass/Unit |
|---|---|---|
| | Ingredient | [mg] |
| Dal | According to example 1 | 571.00 |
| layer | | |
| Atv Layer | API 2 | 5.41 |
| | Lactose Pulvis | 36.09 |
| | Microcrystalline Cellulose (PH 102) | 28.6 |
| | Croscarmellose Sodium | 2.18 |
| | Magnesium carbonate | 17.5 |
| | HPC (Klucel LF) | 1.45 |
| | Polysorbate 80 (Tween 80) | 0.29 |
| | Lactose (Tablettose 70) | 117.2 |
| | Microcrystalline Cellulose (PH 102) | 150.81 |
| | Croscarmellose Sodium | 15.22 |
| | Magnesium stearate | 1.88 |

### Example No 20

| | | Mass/Unit |
|---|---|---|
| | Ingredient | [mg] |
| Dal | According to example 1 | 571.00 |
| layer | | |
| Atv Layer | API 2 | 10.82 |
| | Lactose Pulvis | 72.18 |
| | Microcrystalline Cellulose (PH 102) | 57.19 |
| | Croscarmellose Sodium | 4.35 |
| | Magnesium carbonate | 35 |
| | HPC (Klucel LF) | 2.9 |
| | Polysorbate 80 (Tween 80) | 0.58 |
| | Lactose (Tablettose 70) | 100.7 |
| | Microcrystalline Cellulose (PH 102) | 77.97 |
| | Croscarmellose Sodium | 13.05 |
| | Magnesium stearate | 1.88 |

### Example No 21

| | | Mass/Unit |
|---|---|---|
| | Ingredient | [mg] |
| Dal | According to example 1 | 571.00 |
| layer | | |
| Atv Layer | API 2 | 21.64 |
| | Lactose Pulvis | 144.36 |
| | Microcrystalline Cellulose (PH 102) | 114.38 |
| | Croscarmellose Sodium | 8.7 |
| | Magnesium carbonate | 70 |
| | HPC (Klucel LF) | 5.8 |
| | Polysorbate 80 (Tween 80) | 1.16 |
| | Croscarmellose Sodium | 8.7 |
| | Magnesium stearate | 1.88 |

### Example No 22

| | | Mass/Unit |
|---|---|---|
| | Ingredient | [mg] |
| Dal | According to example 1 | 571.00 |
| layer | | |
| Atv Layer | API 2 | 5.41 |
| | Lactose Pulvis | 3.34 |
| | Microcrystalline Cellulose (PH 102) | 5.93 |
| | Croscarmellose Sodium | 2.18 |
| | Magnesium carbonate | 13.86 |
| | HPC (Klucel LF) | 1.45 |
| | Polysorbate 80 (Tween 80) | 0.29 |
| | Lactose (Tablettose 70) | 32.73 |
| | Microcrystalline Cellulose (PH 102) | 58.12 |
| | Croscarmellose Sodium | 15.23 |
| | Magnesium stearate | 5.41 |

### Example No 23

| | | Mass/Unit |
|---|---|---|
| | Ingredient | [mg] |
| Dal | According to example 1 | 571.00 |
| layer | | |
| Atv Layer | API 2 | 21.65 |
| | Lactose Pulvis | 13.36 |
| | Microcrystalline Cellulose (PH 102) | 23.72 |
| | Croscarmellose Sodium | 8.70 |
| | Magnesium carbonate | 55.45 |
| | HPC (Klucel LF) | 5.80 |
| | Polysorbate 80 (Tween 80) | 1.16 |
| | Croscarmellose Sodium | 8.70 |
| | Magnesium stearate | 1.46 |

### Example No 24

| | | Mass/Unit |
|---|---|---|
| | Ingredient | [mg] |
| Dal | According to example 1 | 571.00 |
| layer | | |
| Atv Layer | API 2 | 43.30 |
| | Lactose Pulvis | 26.72 |
| | Microcrystalline Cellulose (PH 102) | 47.44 |
| | Croscarmellose Sodium | 17.40 |
| | Magnesium carbonate | 110.90 |
| | HPC (Klucel LF) | 11.60 |
| | Polysorbate 80 (Tween 80) | 2.32 |
| | Croscarmellose Sodium | 17.40 |
| | Magnesium stearate | 2.92 |

### Example No 25

| | | Mass/Unit |
|---|---|---|
| | Ingredient | [mg] |
| Dal | According to example 1 | 571.00 |
| layer | | |
| Atv Layer | API 2 | 5.41 |
| | Lactose Pulvis | 34.19 |
| | Microcrystalline Cellulose (PH 102) | 60.72 |
| | Croscarmellose Sodium | 8.70 |
| | Magnesium carbonate | 13.86 |
| | HPC (Klucel LF) | 5.80 |
| | Polysorbate 80 (Tween 80) | 1.16 |

### Example No 26

| | | Mass/Unit |
|---|---|---|
| | Ingredient | [mg] |
| Dal | According to example 1 | 571.00 |
| layer | | |
| Atv Layer | API 2 | 5.41 |
| | Lactose Pulvis | 7.84 |
| | Microcrystalline Cellulose (PH 102) | 13.93 |
| | Croscarmellose Sodium | 2.18 |
| | Magnesium carbonate | 13.86 |
| | HPC (Klucel LF) | 1.45 |
| | Polysorbate 80 (Tween 80) | 0.29 |
| | Lactose (Tablettose 70) | 46.24 |
| | Microcrystalline Cellulose (PH 102) | 82.12 |
| | Croscarmellose Sodium | 15.23 |
| | Magnesium stearate | 1.46 |

### Example No 27

| | | Mass/Unit |
|---|---|---|
| | Ingredient | [mg] |
| Dal | According to example 1 | 571.00 |
| layer | | |
| Atv Layer | API 2 | 21.65 |
| | Lactose Pulvis | 31.37 |
| | Microcrystalline Cellulose (PH 102) | 55.71 |
| | Croscarmellose Sodium | 8.70 |
| | Magnesium carbonate | 55.45 |
| | HPC (Klucel LF) | 5.80 |
| | Polysorbate 80 (Tween 80) | 1.16 |
| | Croscarmellose Sodium | 8.70 |
| | Magnesium stearate | 1.46 |

### Example No 28

| | | Mass/Unit |
|---|---|---|
| | Ingredient | [mg] |
| Dal | According to example 1 | 571.00 |
| layer | | |
| Atv | API 2 | 43.30 |
| Layer | Lactose Pulvis | 62.74 |
| | Microcrystalline Cellulose (PH 102) | 111.42 |
| | Croscarmellose Sodium | 17.40 |
| | Magnesium carbonate | 110.90 |
| | HPC (Klucel LF) | 11.60 |
| | Polysorbate 80 (Tween 80) | 2.32 |
| | Croscarmellose Sodium | 17.40 |
| | Magnesium stearate | 2.92 |

### Example No 29

| | | Mass/Unit |
|---|---|---|
| | Ingredient | [mg] |
| Dal | According to example 1 | 571.00 |
| layer | | |
| Atv | API 2 | 5.41 |
| Layer | Lactose Pulvis | 52.20 |
| | Microcrystalline Cellulose (PH 102) | 92.70 |
| | Croscarmellose Sodium | 8.70 |
| | Magnesium carbonate | 13.86 |
| | HPC (Klucel LF) | 5.80 |
| | Polysorbate 80 (Tween 80) | 1.16 |
| | Croscarmellose Sodium | 8.70 |
| | Magnesium stearate | 1.46 |

### Example No 30

| | | Mass/Unit |
|---|---|---|
| | Ingredient | [mg] |
| Dal | According to example 1 | 571.00 |
| layer | | |
| Atv | API 2 | 5.41 |
| Layer | Lactose Pulvis | 12.35 |
| | Microcrystalline Cellulose (PH 102) | 21.92 |
| | Croscarmellose Sodium | 2.18 |
| | Magnesium carbonate | 13.86 |
| | HPC (Klucel LF) | 1.45 |
| | Polysorbate 80 (Tween 80) | 0.29 |
| | Lactose (Tablettose 70) | 59.75 |
| | Microcrystalline Cellulose (PH 102) | 106.11 |
| | Croscarmellose Sodium | 15.23 |
| | Magnesium stearate | 1.46 |

### Example No 31

| | | Mass/Unit |
|---|---|---|
| | Ingredient | [mg] |
| Dal | According to example 1 | 571.00 |
| layer | | |
| Atv | API 2 | 21.65 |
| Layer | Lactose Pulvis | 49.38 |
| | Microcrystalline Cellulose (PH 102) | 87.70 |
| | Croscarmellose Sodium | 8.70 |
| | Magnesium carbonate | 55.45 |
| | HPC (Klucel LF) | 5.80 |
| | Polysorbate 80 (Tween 80) | 1.16 |
| | Croscarmellose Sodium | 8.70 |
| | Magnesium stearate | 1.46 |

### Example No 32

| | | Mass/Unit |
|---|---|---|
| | Ingredient | [mg] |
| Dal | According to example 1 | 571.00 |
| layer | | |
| Atv | API 2 | 43.30 |
| Layer | Lactose Pulvis | 98.77 |
| | Microcrystalline Cellulose (PH 102) | 175.39 |
| | Croscarmellose Sodium | 17.40 |
| | Magnesium carbonate | 110.90 |
| | HPC (Klucel LF) | 11.60 |
| | Polysorbate 80 (Tween 80) | 2.32 |
| | Croscarmellose Sodium | 17.40 |
| | Magnesium stearate | 2.92 |

### Example No 33

| | | Mass/Unit |
|---|---|---|
| | Ingredient | [mg] |
| Dal | According to example 1 | 571.00 |
| layer | | |
| Atv | Atorvastatin Calcium Trihydrate | 5.40 |
| Layer | Lactose Pulvis | 16.09 |
| | Microcrystalline Cellulose (PH 102) | 28.61 |
| | Croscarmellose Sodium | 2.18 |
| | Calcium carbonate | 15.95 |
| | HPC (Klucel LF) | 1.45 |
| | Polysorbate 80 (Tween 80) | 0.29 |
| | Lactose (Tablettose 70) | 88.93 |
| | Microcrystalline Cellulose (PH 102) | 114.43 |
| | Croscarmellose Sodium | 15.23 |
| | Magnesium stearate | 1.46 |

### Example No 34

| | | Mass/Unit |
|---|---|---|
| | Ingredient | [mg] |
| Dal | According to example 1 | 571.00 |
| layer | | |
| Atv | API 2 | 10.80 |
| Layer | Lactose Pulvis | 32.18 |
| | Microcrystalline Cellulose (PH 102) | 57.22 |
| | Croscarmellose Sodium | 4.35 |
| | Calcium carbonate | 31.90 |
| | HPC (Klucel LF) | 2.90 |
| | Polysorbate 80 (Tween 80) | 0.58 |
| | Lactose (Tablettose 70) | 76.28 |
| | Microcrystalline Cellulose (PH 102) | 59.29 |
| | Croscarmellose Sodium | 13.05 |
| | Magnesium stearate | 1.46 |

### Example No 35

| | | Mass/Unit |
|---|---|---|
| | Ingredient | [mg] |
| Dal | According to example 1 | 571.00 |
| layer | | |
| Atv | Atorvastatin Calcium Trihydrate | 21.60 |
| Layer | Lactose Pulvis | 64.36 |
| | Microcrystalline Cellulose (PH 102) | 114.44 |
| | Croscarmellose Sodium | 8.70 |
| | Calcium carbonate | 63.80 |
| | HPC (Klucel LF) | 5.80 |
| | Polysorbate 80 (Tween 80) | 1.16 |
| | Croscarmellose Sodium | 8.70 |
| | Magnesium stearate | 1.46 |

### Example No 36

| | | Mass/Unit |
|---|---|---|
| | Ingredient | [mg] |
| Dal | According to example 1 | 571.00 |
| layer | | |
| Atv | API 2 | 5.41 |
| Layer | Lactose Pulvis | 2.59 |
| | Microcrystalline Cellulose (PH 102) | 4.60 |
| | Croscarmellose Sodium | 2.18 |
| | Calcium carbonate | 15.95 |
| | HPC (Klucel LF) | 1.45 |
| | Polysorbate 80 (Tween 80) | 0.29 |
| | Lactose (Tablettose 70) | 32.73 |
| | Microcrystalline Cellulose (PH 102) | 58.12 |
| | Croscarmellose Sodium | 15.23 |
| | Magnesium stearate | 1.46 |

### Example No 37

| | | Mass/Unit |
|---|---|---|
| | Ingredient | [mg] |
| Dal | According to example 1 | 571.00 |
| layer | | |
| Atv | Atorvastatin Calcium Trihydrate | 21.65 |
| Layer | Lactose Pulvis | 10.35 |
| | Microcrystalline Cellulose (PH 102) | 18.38 |
| | Croscarmellose Sodium | 8.70 |
| | Calcium carbonate | 63.80 |
| | HPC (Klucel LF) | 5.80 |
| | Polysorbate 80 (Tween 80) | 1.16 |
| | Croscarmellose Sodium | 8.70 |
| | Magnesium stearate | 1.46 |

### Example No 38

| | | Mass/Unit |
|---|---|---|
| | Ingredient | [mg] |
| Dal | According to example 1 | 571.00 |
| layer | | |
| Atv | API 2 | 43.30 |
| Layer | Lactose Pulvis | 20.70 |
| | Microcrystalline Cellulose (PH 102) | 36.76 |
| | Croscarmellose Sodium | 17.40 |
| | Calcium carbonate | 127.60 |
| | HPC (Klucel LF) | 11.60 |
| | Polysorbate 80 (Tween 80) | 2.32 |
| | Croscarmellose Sodium | 17.40 |
| | Magnesium stearate | 2.92 |

### Example No 39

| | | Mass/Unit |
|---|---|---|
| | Ingredient | [mg] |
| Dal | According to example 1 | 571.00 |
| layer | | |
| Atv | API 2 | 5.41 |
| Layer | Lactose Pulvis | 33.44 |
| | Microcrystalline Cellulose (PH 102) | 59.38 |
| | Croscarmellose Sodium | 8.70 |
| | Calcium carbonate | 15.95 |
| | HPC (Klucel LF) | 5.80 |
| | Polysorbate 80 (Tween 80) | 1.16 |
| | Croscarmellose Sodium | 8.70 |
| | Magnesium stearate | 1.46 |

### Example No 40

| | | Mass/Unit |
|---|---|---|
| | Ingredient | [mg] |
| Dal | According to example 1 | 571.00 |
| layer | | |
| Atv | API 2 | 5.41 |
| Layer | Lactose Pulvis | 7.09 |
| | Microcrystalline Cellulose (PH 102) | 12.59 |
| | Croscarmellose Sodium | 2.18 |
| | Calcium carbonate | 15.95 |
| | HPC (Klucel LF) | 1.45 |
| | Polysorbate 80 (Tween 80) | 0.29 |
| | Lactose (Tablettose 70) | 56.13 |
| | Microcrystalline Cellulose (PH 102) | 72.23 |
| | Croscarmellose Sodium | 15.23 |
| | Magnesium stearate | 1.46 |

### Example No 41

| | | Mass/Unit |
|---|---|---|
| | Ingredient | [mg] |
| Dal | According to example 1 | 571.00 |
| layer | | |
| Atv | API 2 | 21.65 |
| Layer | Lactose Pulvis | 28.36 |
| | Microcrystalline Cellulose (PH 102) | 50.37 |
| | Croscarmellose Sodium | 8.70 |
| | Calcium carbonate | 63.80 |
| | HPC (Klucel LF) | 5.80 |
| | Polysorbate 80 (Tween 80) | 1.16 |
| | Croscarmellose Sodium | 8.70 |
| | Magnesium stearate | 1.46 |

### Example No 42

| | | Mass/Unit |
|---|---|---|
| | Ingredient | [mg] |
| Dal | According to example 1 | 571.00 |
| layer | | |
| Atv | API 2 | 43.30 |
| Layer | Lactose Pulvis | 56.72 |
| | Microcrystalline Cellulose (PH 102) | 100.74 |
| | Croscarmellose Sodium | 17.40 |
| | Calcium carbonate | 127.60 |
| | HPC (Klucel LF) | 11.60 |
| | Polysorbate 80 (Tween 80) | 2.32 |
| | Croscarmellose Sodium | 17.40 |
| | Magnesium stearate | 2.92 |

### Example No 43

| | | Mass/Unit |
|---|---|---|
| | Ingredient | [mg] |
| Dal | According to example 1 | 571.00 |
| layer | | |
| Atv | API 2 | 5.41 |
| Layer | Lactose Pulvis | 51.45 |
| | Microcrystalline Cellulose (PH 102) | 91.37 |
| | Croscarmellose Sodium | 8.70 |
| | Calcium carbonate | 15.95 |
| | HPC (Klucel LF) | 5.80 |
| | Polysorbate 80 (Tween 80) | 1.16 |
| | Croscarmellose Sodium | 8.70 |
| | Magnesium stearate | 1.46 |

### Example No 44

| | | Mass/Unit |
|---|---|---|
| | Ingredient | [mg] |
| Dal | According to example 1 | 571.00 |
| layer | | |
| Atv | API 2 | 5.41 |
| Layer | Lactose Pulvis | 11.59 |
| | Microcrystalline Cellulose (PH 102) | 20.59 |
| | Croscarmellose Sodium | 2.18 |
| | Calcium carbonate | 15.95 |
| | HPC (Klucel LF) | 1.45 |
| | Polysorbate 80 (Tween 80) | 0.29 |
| | Lactose (Tablettose 70) | 72.53 |
| | Microcrystalline Cellulose (PH 102) | 93.33 |
| | Croscarmellose Sodium | 15.23 |
| | Magnesium stearate | 1.46 |

### Example No 45

| | | Mass/Unit |
|---|---|---|
| | Ingredient | [mg] |
| Dal | According to example 1 | 571.00 |
| layer | | |
| Atv | API 2 | 21.65 |
| Layer | Lactose Pulvis | 46.37 |
| | Microcrystalline Cellulose (PH 102) | 82.36 |
| | Croscarmellose Sodium | 8.70 |
| | Calcium carbonate | 63.80 |
| | HPC (Klucel LF) | 5.80 |
| | Polysorbate 80 (Tween 80) | 1.16 |
| | Croscarmellose Sodium | 8.70 |
| | Magnesium stearate | 1.46 |

### Example No 46

| | | Mass/Unit |
|---|---|---|
| | Ingredient | [mg] |
| Dal | According to example 1 | 571.00 |
| layer | | |
| Atv | API 2 | 43.30 |
| Layer | Lactose Pulvis | 92.74 |
| | Microcrystalline Cellulose (PH 102) | 164.72 |
| | Croscarmellose Sodium | 17.40 |
| | Calcium carbonate | 127.60 |
| | HPC (Klucel LF) | 11.60 |
| | Polysorbate 80 (Tween 80) | 2.32 |
| | Croscarmellose Sodium | 17.40 |
| | Magnesium stearate | 2.92 |

Not yet produced

### Example No 47

| | | Mass/Unit |
|---|---|---|
| | Ingredient | [mg] |
| Core | According to example 1 | 571.00 |
| Separation | HPMC (2910 3cp) | 5.00 |
| Layer | HPC (Klucel LF) | 5.00 |
| | triacetin | 1.00 |
| | talcum | 9.00 |
| Active | API 2 | 10.83 |
| Coating | Triethylcitrate | 0.5 |
| | HPMC (2910 3cp) | 2.50 |
| | HPC (Klucel LF) | 2.50 |
| | Talcum | 4.5 |
| | Simethicone Suspension USP (30% solids) | 1.20 (0.36)¹ |
| Seal | HPMC (2910 3cp) | 5.00 |
| Coat | HPC (Klucel LF) | 5.00 |
| | triethylcitrate | 1.00 |
| | Talcum | 4.50 |
| | Titan dioxide | 4.50 |

¹In suspension (solvent free)

### Example No 48

| | | Mass/Unit |
|---|---|---|
| | Ingredient | [mg] |
| Core | According to example 1 | 571.00 |
| Separation | HPMC (2910 3cp) | 5.00 |
| Layer | HPC (Klucel LF) | 5.00 |
| | Triacetin | 1.00 |
| | Talcum | 9.00 |
| Active | API 2 | 21.65 |
| Coating | Triethylcitrate | 1.0 |
| | HPMC (2910 3cp) | 5.00 |
| | HPC (Klucel LF) | 5.00 |
| | Talcum | 9.00 |
| | Simethicone Suspension USP (30% solids) | 2.40 (0.72)¹ |
| Seal | HPMC (2910 3cp) | 5.00 |
| Coat | HPC (Klucel LF) | 5.00 |
| | triethylcitrate | 1.00 |
| | Talcum | 4.50 |
| | Titan dioxide | 4.50 |

¹In suspension (solvent free)

### Example No 49

| | | Mass/Unit |
|---|---|---|
| | Ingredient | [mg] |
| Core | According to example 1 | 571.00 |
| Separation | Polyvinyl alcohol (PVP VA 64: | 10.00 |
| Layer | Kollidon VA 64) | |
| | Triacetin | 1.00 |
| | Talcum | 9.00 |
| Active | API 2 | 10.83 |
| Coating | Simethicone Suspension USP (30% solids) | 1.20 (0.36)¹ |
| | Polyvinyl alcohol (PVP VA 64: | 5.00 |
| | Kollidon VA 64) | |
| | Triethylcitrate | 0.50 |
| | Talcum | 4.50 |
| Seal | Polyvinyl alcohol (PVP VA 64: | 10.00 |
| Coat | Kollidon VA 64) | |
| | Triethylcitrate | 1.00 |
| | Talcum | 4.50 |
| | Titan dioxide | 4.50 |

¹In suspension (solvent free)

### Example No 50

| | | Mass/Unit |
|---|---|---|
| | Ingredient | [mg] |
| Core | According to example 1 | 571.00 |
| Separation | Polyvinyl alcohol (PVP VA 64: | 10.00 |
| Layer | Kollidon VA 64) | |
| | Triacetin | 1.00 |
| | Talcum | 9.00 |
| Active | API 2 | 21.65 |
| Coating | Simethicone Suspension USP (30% solids) | 2.40 (0.72)¹ |
| | Polyvinyl alcohol (PVP VA 64: | 10.00 |
| | Kollidon VA 64) | |
| | Triethylcitrate | 1.00 |
| | Talcum | 9.00 |
| Seal | Polyvinyl alcohol (PVP VA 64: | 10.00 |
| Coat | Kollidon VA 64) | |
| | Triethylcitrate | 1.00 |
| | Talcum | 4.50 |
| | Titan dioxide | 4.50 |

¹In suspension (solvent free)

### Example No 51

| | | Mass/Unit |
|---|---|---|
| | Ingredient | [mg] |
| Core | According to example 1 | 571.00 |
| Separation | Kollicoat IR | 10.50 |
| Layer | Talcum | 9.50 |
| Active | API 2 | 10.83 |
| Coating | Simethicone Suspension USP (30% solids) | 1.20 (0.36)¹ |
| | Kollicoat IR | 5.26 |
| | Talcum | 4.76 |
| Seal | Kollicoat IR | 10.50 |
| Coatt | Talcum | 4.75 |
| | Titan dioxide | 4.75 |

¹In suspension (solvent free)

### Example No 52

| | | Mass/Unit |
|---|---|---|
| | Ingredient | [mg] |
| Core | According to example 1 | 571.00 |
| Separation | Kollicoat IR | 10.50 |
| Layer | Talcum | 9.50 |
| Active | API 2 | 21.65 |
| Coating | Simethicone Suspension USP (30% solids) | 2.40 (0.72)¹ |
| | Kollicoat IR | 10.52 |
| | Talcum | 9.52 |
| Seal | Kollicoat IR | 10.50 |
| Coat | Talcum | 4.75 |
| | Titan dioxide | 4.75 |

¹In suspension (solvent free)

### Example No 53

| | | Mass/Unit |
|---|---|---|
| | Ingredient | [mg] |
| Core | According to example 1 | 571.00 |
| Separation | polyvinyl alcohol (PVA EG -05 PW) | 10.00 |
| Layer | triacetin | 1.00 |
| | talcum | 9.00 |
| Active | API 2 | 10.82 |
| Coating | polyvinyl alcohol (PVA EG -05 PW) | 5.00 |
| | CMC Na | 0.10 |
| | Triacetin | 0.50 |
| | Talcum | 4.50 |
| | Simethicone Suspension USP (30% solids) | 1.20 (0.36)¹ |
| Seal | polyvinyl alcohol (PVA EG -05 PW) | 10.00 |
| Coat | Triacetin | 1.00 |
| | Talcum | 4.50 |
| | Titan dioxide | 4.50 |

¹In suspension (solvent free)

### Example No 54

| | | Mass/Unit |
|---|---|---|
| | Ingredient | [mg] |
| Core | According to example 1 | 571.00 |
| Separation | polyvinyl alcohol (PVA EG -05 PW) | 10.00 |
| Layer | triacetin | 1.00 |
| | talcum | 9.00 |
| Active | API 2 | 21.65 |
| Coating | polyvinyl alcohol (PVA EG -05 PW) | 10.00 |
| | CMC Na | 0.05 |
| | Triacetin | 1.00 |
| | Talcum | 9.00 |
| | Simethicone Suspension USP (30% solids) | 2.40 (0.72)¹ |
| Seal | polyvinyl alcohol (PVA EG -05 PW) | 10.00 |
| Coat | Triacetin | 1.00 |
| | Talcum | 4.50 |
| | Titan dioxide | 4.50 |

¹In suspension (solvent free)

### Example No 55

| | | Mass/Unit |
|---|---|---|
| | Ingredient | [mg] |
| Core | According to example 1 | 571.00 |
| Separation | Opadry II white 85F18422 | 20.00 |
| Layer | | |
| Active | API 2 | 43.2 |
| Coating | Opadry II clear 85F29116 | 159.6 |
| | Calcium carbonate | 129.6 |
| | Simethicone Suspension USP (30% solids) | 3.6 (1.08)¹ |
| Seal | Opadry II brown 85F26792 | 15.00 |
| Coat | | |

¹In suspension (solvent free)

### Example No 56

| | | Mass/Unit |
|---|---|---|
| | Ingredient | [mg] |
| Core | According to example 1 | 571.00 |
| Separation | polyvinyl alcohol (PVA EG -05 PW) | 10.00 |
| Layer | Triacetin | 1.00 |
| | Talcum | 9.00 |
| Active | API 2 | 10.82 |
| Coating | polyvinyl alcohol (PVA EG -05 PW) | 5.00 |
| | Triacetin | 0.50 |
| | Talcum | 4.50 |
| | Simethicone Suspension USP (30% solids) | 1.2 (0.36)¹ |
| Seal | polyvinyl alcohol (PVA EG -05 PW) | 10.00 |
| Coat | Triacetin | 1.00 |
| | Talcum | 4.50 |
| | Titan dioxide | 4.50 |

¹In suspension (solvent free)

### Example No 57

| | | Mass/Unit |
|---|---|---|
| | Ingredient | [mg] |
| Core | According to example 1 | 571.00 |
| Separation | polyvinyl alcohol (PVA EG -05 PW) | 10.00 |
| Layer | Triacetin | 1.00 |
| | Talcum | 9.00 |
| Active | API 2 | 21.64 |
| Coating | polyvinyl alcohol (PVA EG -05 PW) | 10.00 |
| | Triacetin | 1.00 |
| | Talcum | 9.00 |
| | Simethicone Suspension USP (30% solids) | 2.4 (0.72)¹ |
| Seal | polyvinyl alcohol (PVA EG -05 PW) | 10.00 |
| Coat | Triacetin | 1.00 |
| | Talcum | 4.50 |
| | Titan dioxide | 4.50 |

¹In suspension (solvent free)

### Example No 58

| | | Mass/Unit |
|---|---|---|
| | Ingredient | [mg] |
| Core | According to example 1 | 571.00 |
| Separation | polyvinyl alcohol (PVA EG -05 PW) | 10.00 |
| | Triacetin | 1.00 |
| | Talcum | 9.00 |
| Active | API 2 | 10.82 |
| Coating | polyvinyl alcohol (PVA EG -05 PW) | 20.00 |
| | Triacetin | 2.00 |
| | Talcum | 18.00 |
| | Simethicone Suspension USP (30% solids) | 4.8 (1.44)¹ |
| Seal | polyvinyl alcohol (PVA EG -05 PW) | 10.00 |
| Coat | Triacetin | 1.00 |
| | Talcum | 4.50 |
| | Titan dioxide | 4.50 |

¹In suspension (solvent free)

### Example No 59

| | | Mass/Unit |
|---|---|---|
| | Ingredient | [mg] |
| Core | According to example 1 | 571.00 |
| Separation | polyvinyl alcohol (PVA EG -05 PW) | 10.00 |
| Layer | Triacetin | 1.00 |
| | Talcum | 9.00 |
| Active | API 2 | 21.65 |
| Coating | polyvinyl alcohol (PVA EG -05 PW) | 20.00 |
| | Triacetin | 2.00 |
| | Talcum | 18.00 |
| | Simethicone Suspension USP (30% solids) | 4.8 (1.44)¹ |
| Seal | polyvinyl alcohol (PVA EG -05 PW) | 10.00 |
| Coat | Triacetin | 1.00 |
| | Talcum | 4.50 |
| | Titan dioxide | 4.50 |

¹In suspension (solvent free)

### Example No 60

| | | Mass/Unit |
|---|---|---|
| | Ingredient | [mg] |
| Dal | According to example 1 | 571.00 |
| layer | | |
| Atv | API 2 | 43.28 |
| Layer | Lactose Pulvis | 208.72 |
| | Microcrystalline Cellulose (PH 102) | 228.76 |
| | Croscarmellose Sodium | 17.40 |
| | Magnesium oxide | 120.00 |
| | HPC (Klucel LF) | 11.60 |
| | Polysorbate 80 (Tween 80) | 2.32 |
| | Croscarmellose Sodium | 17.46 |
| | Magnesium stearate | 5.18 |

### Example No 61

| | | Mass/Unit |
|---|---|---|
| | Ingredient | [mg] |
| Dal | According to example 1 | 571.00 |
| layer | | |
| Atv | API 2 | 43.28 |
| Layer | Lactose Pulvis | 288.72 |
| | Microcrystalline Cellulose (PH 102) | 228.76 |
| | Croscarmellose Sodium | 17.4 |
| | Magnesium carbonate | 140 |
| | HPC (Klucel LF) | 11.6 |
| | Polysorbate 80 (Tween 80) | 2.32 |
| | Croscarmellose Sodium | 17.4 |
| | Magnesium stearate | 3.76 |

### Example No 62

| | | Mass/Unit |
|---|---|---|
| | Ingredient | [mg] |
| Dal | According to example 1 | 571.00 |
| layer | | |
| Atv | API 2 | 10.83 |
| Layer | Lactose Pulvis | 6.68 |
| | Microcrystalline Cellulose (PH 102) | 11.86 |
| | Croscarmellose Sodium | 4.35 |
| | Magnesium carbonate | 27.73 |
| | HPC (Klucel LF) | 2.90 |
| | Polysorbate 80 (Tween 80) | 0.58 |
| | Lactose (Tablettose 70) | 34.14 |
| | Microcrystalline Cellulose (PH 102) | 26.43 |
| | Croscarmellose Sodium | 13.05 |
| | Magnesium stearate | 1.46 |

### Example No 63

| | | Mass/Unit |
|---|---|---|
| | Ingredient | [mg] |
| Dal | According to example 1 | 571.00 |
| layer | | |
| Atv | API 2 | 10.83 |
| Layer | Lactose Pulvis | 15.69 |
| | Microcrystalline Cellulose (PH 102) | 27.85 |
| | Croscarmellose Sodium | 4.35 |
| | Magnesium carbonate | 27.73 |
| | HPC (Klucel LF) | 2.90 |
| | Polysorbate 80 (Tween 80) | 0.58 |
| | Lactose (Tablettose 70) | 48.23 |
| | Microcrystalline Cellulose (PH 102) | 37.34 |
| | Croscarmellose Sodium | 13.05 |
| | Magnesium stearate | 1.46 |

### Example No 64

| | | Mass/Unit |
|---|---|---|
| | Ingredient | [mg] |
| Dal | According to example 1 | 571.00 |
| layer | | |
| Atv | API 2 | 43.20 |
| Layer | Lactose Pulvis | 128.72 |
| | Microcrystalline Cellulose (PH 102) | 228.88 |
| | Croscarmellose Sodium | 17.40 |
| | Calcium carbonate | 127.60 |
| | HPC (Klucel LF) | 11.60 |
| | Polysorbate 80 (Tween 80) | 2.32 |
| | Croscarmellose Sodium | 17.40 |
| | Magnesium stearate | 2.92 |

### Example No 65

| | | Mass/Unit |
|---|---|---|
| | Ingredient | [mg] |
| Dal | According to example 1 | 571.00 |
| layer | | |
| Atv | API 2 | 10.83 |
| Layer | Lactose Pulvis | 5.18 |
| | Microcrystalline Cellulose (PH 102) | 9.19 |
| | Croscarmellose Sodium | 4.35 |
| | Calcium carbonate | 31.90 |
| | HPC (Klucel LF) | 2.90 |
| | Polysorbate 80 (Tween 80) | 0.58 |
| | Lactose (Tablettose 70) | 34.08 |
| | Microcrystalline Cellulose (PH 102) | 26.49 |
| | Croscarmellose Sodium | 13.05 |
| | Magnesium stearate | 1.46 |

### Example No 66

| | | Mass/Unit |
|---|---|---|
| | Ingredient | [mg] |
| Dal | According to example 1 | 571.00 |
| layer | | |
| Atv | API 2 | 10.83 |
| Layer | Lactose Pulvis | 25.72 |
| | Microcrystalline Cellulose (PH 102) | 45.74 |
| | Croscarmellose Sodium | 8.70 |
| | Calcium carbonate | 31.90 |
| | HPC (Klucel LF) | 5.80 |
| | Polysorbate 80 (Tween 80) | 1.16 |
| | Croscarmellose Sodium | 8.70 |
| | Magnesium stearate | 1.46 |

### Example No 67

| | | Mass/Unit |
|---|---|---|
| | Ingredient | [mg] |
| Dal | According to example 1 | 571.00 |
| layer | | |
| Atv | API 2 | 10.83 |
| Layer | Lactose Pulvis | 14.18 |
| | Microcrystalline Cellulose (PH 102) | 25.19 |
| | Croscarmellose Sodium | 4.35 |
| | Calcium carbonate | 31.90 |
| | HPC (Klucel LF) | 2.90 |
| | Polysorbate 80 (Tween 80) | 0.58 |
| | Lactose (Tablettose 70) | 48.15 |
| | Microcrystalline Cellulose (PH 102) | 37.42 |
| | Croscarmellose Sodium | 13.05 |
| | Magnesium stearate | 1.46 |

### Example No 68

| | | Mass/Unit |
|---|---|---|
| | Ingredient | [mg] |
| Dal | According to example 1 | 571.00 |
| layer | | |
| Atv | API 2 | 10.83 |
| Layer | Lactose Pulvis | 43.72 |
| | Microcrystalline Cellulose (PH 102) | 77.74 |
| | Croscarmellose Sodium | 8.70 |
| | Calcium carbonate | 31.90 |
| | HPC (Klucel LF) | 5.80 |
| | Polysorbate 80 (Tween 80) | 1.16 |
| | Croscarmellose Sodium | 8.70 |
| | Magnesium stearate | 1.46 |

### Example No 69

| | | Mass/Unit |
|---|---|---|
| | Ingredient | [mg] |
| Dal | According to example 1 | 571.00 |
| layer | | |
| Atv | API 2 | 10.83 |
| Layer | Lactose Pulvis | 23.19 |
| | Microcrystalline Cellulose (PH 102) | 41.18 |
| | Croscarmellose Sodium | 4.35 |
| | Calcium carbonate | 31.90 |
| | HPC (Klucel LF) | 2.90 |
| | Polysorbate 80 (Tween 80) | 0.58 |
| | Lactose (Tablettose 70) | 62.21 |
| | Microcrystalline Cellulose (PH 102) | 48.36 |
| | Croscarmellose Sodium | 13.05 |
| | Magnesium stearate | 1.46 |

### Example No 70

| | | Mass/Unit |
|---|---|---|
| | Ingredient | [mg] |
| Dal | According to example 1 | 571.00 |
| layer | | |
| Atv | API 2 | 10.83 |
| Layer | Lactose Pulvis | 61.72 |
| | Microcrystalline Cellulose (PH 102) | 109.74 |
| | Croscarmellose Sodium | 8.70 |
| | Calcium carbonate | 31.90 |
| | HPC (Klucel LF) | 5.80 |
| | Polysorbate 80 (Tween 80) | 1.16 |
| | Croscarmellose Sodium | 8.70 |
| | Magnesium stearate | 1.46 |

### Example No A

| | | | hygroscopic polymer | |
|---|---|---|---|---|
| | Mass/Unit | Amount/Unit | Water | Water |
| Ingredient | [mg] | [%] | insoluble | soluble |
| Mannitol | 300.00 | 52.54 | N/A | N/A |
| Microcrystalline | 141.00 | 24.69 | + | |
| Cellulose | | | | |
| Crospovidone | 66.00 | 11.56 | + | |
| micronized | | | | |
| HPMC | 24.00 | 4.20 | | + |
| CMC Na | 34.00 | 5.95 | + | |
| Colloidal Silicon | 3.00 | 0.53 | N/A | N/A |
| Dioxide | | | | |
| Sodium | 3.00 | 0.53 | N/A | N/A |
| stearylfumarate | | | | |
| Total tablet | 571.00 | 100.00 | | |
| Amount hygroscopic | | | 42.21% | 4.20% |
| polymers | | | | |
| Total amount | | | 46.41% | |
| hygroscopic polymers | | | | |

### Example B:

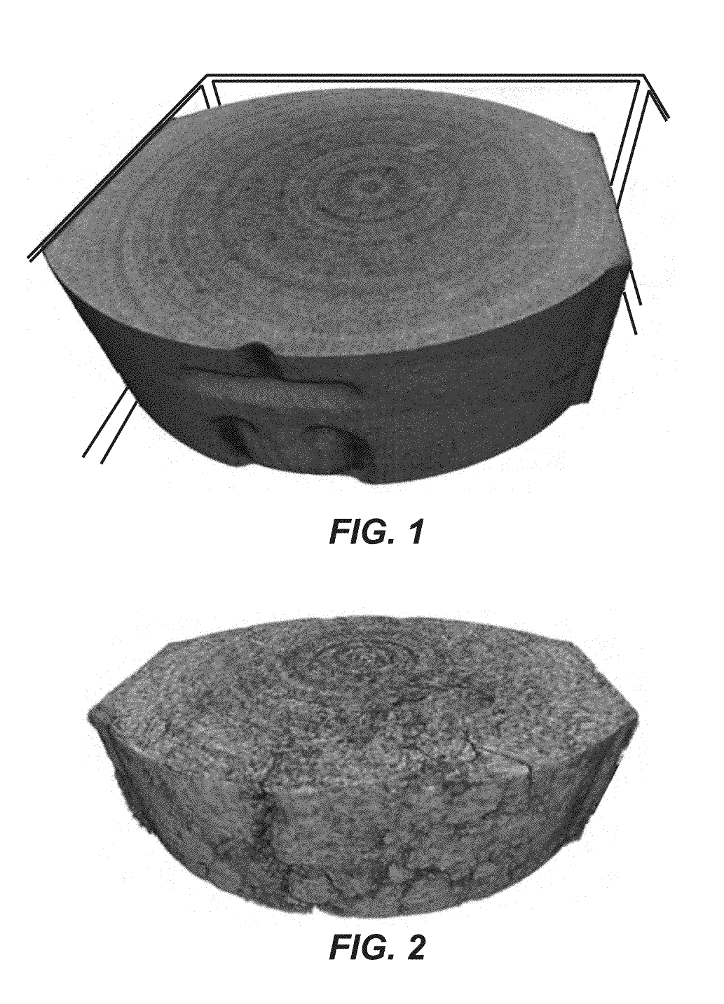

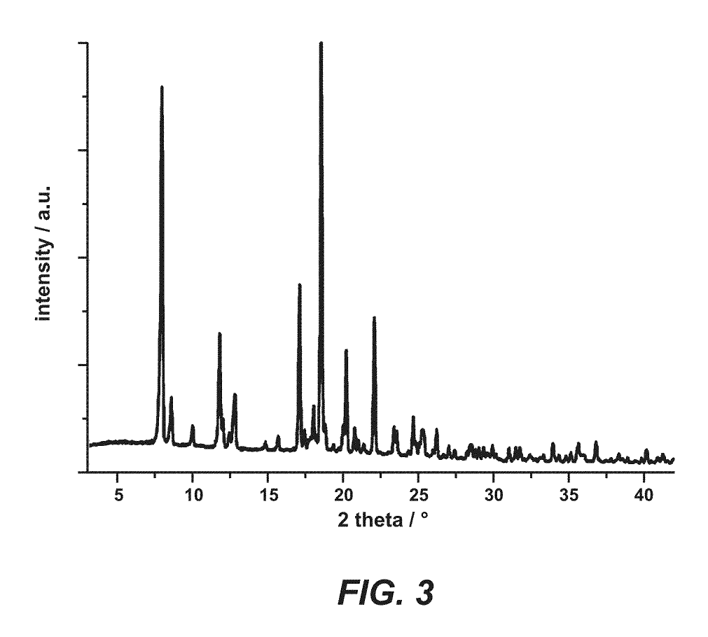

Two tablets produced according to example 1 and example A were sliced and their X-ray pictures were collected. Both figures represent an overlayed of all X-ray slices that were generated during the measurement to reconstruct the 3D tablet. While FIG. 1 does not show any imperfection but a smooth surface, FIG. 2 has lot of cracks. These cracks are also detectable by the human eye.

### Example C

XRPD patterns of S-[2-([[1-(2-ethylbutyl)cyclohexyl]carbonyl]amino) phenyl] 2-methylpropanethioate crystalline form A were recorded at ambient conditions in transmission geometry with a STOE STADI P diffractometer (Cu K alpha radiation source, primary monochromator, position sensitive detector, angular range 3° to 42° 2Theta, approximately 60 minutes total measurement time). The samples were prepared and analyzed without further processing (e.g. grinding or sieving) of the substance.

| | relative |
|---|---|
| 2theta/° | intensity/% |
| 7.9 | 86.3 |
| 8.5 | 16.2 |
| 11.7 | 30.7 |
| 12.7 | 17.1 |
| 17.1 | 41.6 |
| 18 | 14.6 |
| 18.5 | 100 |
| 20.2 | 27.2 |
| 22.1 | 33.7 |
| 24.7 | 11.9 |

1. A composition comprising:
a) S-[2-([[1-(2-ethylbutyl)-cyclohexyl]-carbonyl]amino)phenyl]2-methylpropanethioate;
croscarmellose sodium; and
b) atorvastatin.

2. The composition of claim 1, comprising:
a) a core or one layer comprising:
S-[2-([[1-(2-ethylbutyl)-cyclohexyl]-carbonyl]amino)phenyl]2-methylpropanethioate;
croscarmellose sodium; and
b) an active coating or another layer comprising atorvastatin.

3. The composition of claim 1, comprising:
a) a core comprising:
S-[2-([[1-(2-ethylbutyl)-cyclohexyl]-carbonyl]amino)phenyl]2-methylpropanethioate;
croscarmellose sodium; and
b) an active coating comprising atorvastatin.

4. The composition of claim 1, comprising:
a) one layer comprising:
S-[2-([[1-(2-ethylbutyl)-cyclohexyl]-carbonyl]amino)phenyl]2-methylpropanethioate;
croscarmellose sodium; and
b) another layer comprising atorvastatin.

5. The composition of claim 1, further comprising at least one additional hygroscopic polymeric excipient, in particular wherein the hygroscopic polymeric excipient is in the core or dal layer.

6. The composition of claim 1, further comprising at least two hygroscopic polymeric excipients, in particular wherein the hygroscopic polymeric excipients are in the core or dal layer.

7. The composition of claim 1, further comprising at least three additional hygroscopic polymeric excipients of which two are diluents with a bulk density lower than 800 g/L, in particular wherein the hygroscopic polymeric excipients are in the core or dal layer.

8. The composition of claim 1, comprising:
a) more than 50% by weight of the total weight of the core or dal layer of S-[2-([[1-(2-ethylbutyl)-cyclohexyl]-carbonyl]amino)phenyl]2-methylpropanethioate;
more than 40% by weight of the total weight of the core or dal layer of water insoluble hygroscopic polymer;
more than 4% by weight of the total weight of the core or dal layer of water soluble hygroscopic polymer;
less than 6% of other excipients; and
b) atorvastatin.

9. The composition of claim 1, comprising:
a) 48% to 55% by weight of the total weight of the core or dal layer of S-[2-([[1-(2-ethylbutyl)-cyclohexyl]-carbonyl]amino)phenyl]2-methylpropanethioate;
4% to 8% by weight of the total weight of the core or dal layer of croscarmellose sodium;
32% to 41% by weight of the total weight of the core or dal layer of water insoluble hygroscopic polymer;
4% to 5% by weight of the total weight of the core or dal layer of water soluble hygroscopic polymer; and
b) atorvastatin.

10. The composition of claim 1, wherein the hygroscopic polymeric excipients are selected from hydroxypropylmethyl cellulose, hydroxypropyl cellulose, low-substituted hydroxypropyl cellulose, hydroxyethylmethyl cellulose, carboxypolymethylene, methylcellulose, ethylcellulose, hydroxyethyl cellulose, celluloseacetate, polyvinylpyrrolidone, crosslinked polyvinylpyrrolidone, micronized crosslinked polyvinylpyrrolidone, carboxymethylcellulose calcium, crosslinked carboxymethylcellulose, microcrystalline cellulose, silicified microcrystalline cellulose, cellulose powder, carboxymethyl starch, starch, pregelatinized starch.

11. The composition of claim 1, wherein S-[2-([[1-(2-ethylbutyl)-cyclohexyl]-carbonyl]amino)phenyl]2-methylpropanethioate is in crystalline form.

12. The composition of claim 1, wherein the active coating comprises:
a) atorvastatin;
b) Polyvinyl alcohol;
c) croscarmellose sodium;
d) triacetin;
e) talcum; and
f) simethicone.

13. The composition of claim 2, wherein the active coating is separated by a separation layer from the core:

14. The composition of claim 2, wherein another layer comprising atorvastatin comprises:
atorvastatin;
Lactose;
Microcrystalline Cellulose;
Croscarmellose Sodium;
Magnesium carbonate, calcium carbonate or magnesium oxide;
hydroxypropyl cellulose (HPC);
Polysorbate 80; and
Magnesium stearate.

15. The composition of claim 1, wherein atorvastatin is [R-(R*,R*)]-2-(4-fluorophenyl)-β,δ-dihydroxy-5-(1-methylethyl)-3-phenyl-4-[(phenylamino)carbonyl]-1H-pyrrole-1-heptanoic acid, calcium salt (2:1) trihydrate, most particularly in crystalline form I.

16. The composition of claim 2, wherein the hygroscopic polymeric excipients are hydroxypropylmethyl cellulose, microcrystalline cellulose and micronized crosslinked polyvinylpyrrolidone.

17. The composition of claim 1, comprising:
b) S-[2-([[1-(2-ethylbutyl)-cyclohexyl]-carbonyl]amino)phenyl]2-methylpropanethioate;
microcrystalline cellulose;
crospovidone micronized;
hydroxypropylmethyl cellulose;
croscarmellose sodium; and
b) atorvastatin.

18. The composition of claim 1, comprising:
a) 48% to 55% by weight of the total weight of the core or dal layer of -[2-([[1-(2-ethylbutyl)-cyclohexyl]-carbonyl]amino)phenyl]2-methylpropanethioate;
4% to 8% by weight of croscarmellose sodium;
35% to 44% by weight of hydroxypropylmethyl cellulose, microcrystalline cellulose and crospovidone micronized; and
b) atorvastatin.

19. The composition of claim 1, comprising 10% to 69% by weight of the total weight of the core or dal layer of S-[2-([[1-(2-ethylbutyl)-cyclohexyl]-carbonyl]amino)phenyl]2-methylpropanethioate.

20. A method for the for the treatment or prevention of a cardiovascular disorder, the method comprising administering an effective amount of a composition of claim 1.
